SEQUENCE LISTING (40 sequences)

SEQ ID NO: 1 - DNA, 24 bp, Artificial Sequence
  forward primer
ttattagcgggttagacgtcgttt

SEQ ID NO: 2 - DNA, 18 bp, Artificial Sequence
  reverse primer
cgtcccgaaatcgaaccc

SEQ ID NO: 3 - DNA, 30 bp, Artificial Sequence
  probe
cttactctccgctcccaattcgaaaaatcc

SEQ ID NO: 4 - DNA, 18 bp, Artificial Sequence
  forward primer
agtcgggcggttcggtta

SEQ ID NO: 5 - DNA, 20 bp, Artificial Sequence
  reverse primer
ccaaaaacgacgcgcaacta

SEQ ID NO: 6 - DNA, 17 bp, Artificial Sequence
  probe
cccaactaacgcgctaa

SEQ ID NO: 7 - DNA, 21 bp, Artificial Sequence
  forward primer
ggttattttgcgagcggtttc

SEQ ID NO: 8 - DNA, 24 bp, Artificial Sequence
  reverse primer
cgaatactctaattccacgcgact

SEQ ID NO: 9 - DNA, 28 bp, Artificial Sequence
  probe
aaccgcgtcgaaaaacgaaaacgactac

SEQ ID NO: 10 - DNA, 26 bp, Artificial Sequence
  forward primer
cgcgtagtattaagtagagaggttcg

SEQ ID NO: 11 - DNA, 27 bp, Artificial Sequence
  reverse primer
atctacaataaaaaataaaaccgaccg

SEQ ID NO: 12 - DNA, 27 bp, Artificial Sequence
  probe
ccactaaaccgacgctaattcgacgct

SEQ ID NO: 13 - DNA, 24 bp, Artificial Sequence
  forward primer
gtttggtttttgcggtttttattc

SEQ ID NO: 14 - DNA, 21 bp, Artificial Sequence
  reverse primer
caaccctacctcgcatttacg

SEQ ID NO: 15 - DNA, 27 bp, Artificial Sequence
  probe
ctcgatccaattccatctcgcacttcc

SEQ ID NO: 16 - DNA, 19 bp, Artificial Sequence
  forward primer
gggcgggttaatgagttgc

SEQ ID NO: 17 - DNA, 22 bp, Artificial Sequence
  reverse primer
tcacgtactaccgacgctaacg

SEQ ID NO: 18 - DNA, 19 bp, Artificial Sequence
  probe
cgccgcgccaacgaaaaac

SEQ ID NO: 19 - DNA, 21 bp, Artificial Sequence
  forward primer
gcgttggttgcgttgtttatc

SEQ ID NO: 20 - DNA, 29 bp, Artificial Sequence
  reverse primer
ctacaaaaactaacgaaatctaaaatccg

SEQ ID NO: 21 - DNA, 19 bp, Artificial Sequence
  probe
ccgataacacaacccaaaa

SEQ ID NO: 22 - DNA, 21 bp, Artificial Sequence
  forward primer
gcggagtggtgagattgagtc

SEQ ID NO: 23 - DNA, 25 bp, Artificial Sequence
  reverse primer
aaaatataccgacttcttcgattcg

SEQ ID NO: 24 - DNA, 32 bp, Artificial Sequence
  probe
cctaaaaccgaacaaactaaatctccccaacg

SEQ ID NO: 25 - DNA, 27 bp, Artificial Sequence
  forward primer
gttgtgatattgcgattagaatttgtc

SEQ ID NO: 26 - DNA, 32 bp, Artificial Sequence
  reverse primer
aacgaatcattaattataaaaaattactaccg

SEQ ID NO: 27 - DNA, 40 bp, Artificial Sequence
  probe
acaaatacccaacaaaaataatattttaacaacccgattc

SEQ ID NO: 28 - DNA, 21 bp, Artificial Sequence
  forward primer
gaggtgataggcgtgttggtc

SEQ ID NO: 29 - DNA, 22 bp, Artificial Sequence
  reverse primer
caaccttaaacaaatccgaacg

SEQ ID NO: 30 - DNA, 32 bp, Artificial Sequence
  probe
gcctcgcgactacgctcgatataaatattaac

SEQ ID NO: 31 - DNA, 1700 bp, Homo sapiens
cagcctcacagcctcttcttcacttgccgcctccacacacctccaaccctgcctttttcctttctcctgttccctccctgaccaacctcggcaccccgccaccaggtttgcatcaagcaaaagtaatataccctacagcagactctctaccactaccgagtgatacaaaggactgggattctggacagggtttactcacatatcttgcccctgtgcgctcagaagtcccaaccgggtgggagatccgacactggtccccgagtccccagctcgctgtgtgaccttggacaagtagtcacatcgctgaacctgggtcctataactgcgtgagactattctttggtgctcaccggtatgaatacaaacacaaggtccttcttttctctctctatcccccttcccaagtcgagttggtcaagctcgctttttctttccaccagggccatagccagctgcagccttgtggaggaagaaatcaggtcccagctgctaaaggtgtcctccccaccgccaccgtcgtcttcacccccacccccgggtgtgacctgtccttccggggcacactagtccagcgtcgcccagggcttcggagagagcgcctcacgctcccgcttcgcggcgcctggtccctgcggtccccactcgctgcgacgctttgggaagtgcgagatggaactggatcgagaacgcaaatgcgaggcagggctggtgacaggtatgcgtgtgggcgccggccggaggatgtgttgggagaaactttgggggagttggtgggggaaaggggaggtgtcagagaaagcagtgggtcactctctcacccactgaccaccagtctctctccctcttccttgtccccatctcaccttcttcctcggcttctgcctctcacctccctctctttcttctccaagcatcctccctacgcgtctgcacccgctcctccctcgcaccctcccgcgcctaagcggacctcctcgggagccagctcggtccagcctcccagcgcagtcacgtcccagagcctgttcagctgagccggcagcatgtggaacgcgacgcccagcgaagagccggggttcaacctcacactggccgacctggactgggatgcttcccccggcaacgactcgctgggcgacgagctgctgcagctcttccccgcgccgctgctggcgggcgtcacagccacctgcgtggcactcttcgtggtgggcatcgctggcaacctgctcaccatgctggtggtgtcgcgcttccgcgagctgcgcaccaccaccaacctctacctgtccagcatggccttctccgatctgctcatcttcctctgcatgcccctggacctcgttcgcctctggcagtaccggccctggaacttcggcgacctcctctgcaaactcttccaattcgtcagtgagagctgcacctacgccacggtgctcaccatcacagcgctgagcgtcgagcgctacttcgccatctgcttcccactccgggccaaggtggtggtcaccaaggggcgggtgaagctggtcatcttcgtcatctgggccgtggccttctgcagcgccgggcccatcttcgtgctagtcggggtggagcacgagaacggcaccgacccttgggacaccaacgagtgccgccccaccgagtttgcggtgcgctctggactgctcacggtcatggtgtgggtgtccagcatc

SEQ ID NO: 32 - DNA, 1750 bp, Homo sapiens
tctaagctggaggttgcggggccaattcccttttggcaggcgagaatccggactcgggcggatttcaggcttcagtgtttgtaggaggaaacacagcaatcacactattaatagtaaattaaaataaatgggcaactgctgcatggtaatacttttttttttaaggcaaaaaataaaaaatagtgaaacagagaaacaaaacatgaaacaccggcagtcaacaggcaggcaaagaacctgggggtgggggtagcagcggtcccaccctcaaaaggcccgggctgcccagaccaagagaaagcgatgaatctcttctggtaacgtcccttcctgtcgcatggattcaaggccgacctgccccagcaccaccaccagcagccttctgctggggccggcacagctgggagcaacctcctactctcaggcagacgcgcagcaccaagcagagaggcccggtgcaggatcccagcgccgaaccagcgccggctcagtggacgcggaaggggccggcggccgcggccggtcccatcccccactgcagacccccagcctgtggcggtggtccagttccgccaggaaaccgccgcctggagctgtgggtcgcgcacattaacgcatccagcggaaaaatgaaggagacccaaattcaaagttaaagtaatggtgacccgagaggtgccttgatgagaaggtttggggtcccggttactgatggttatcattcttacgagatgctggtcacctacgaagggagaaaggcacgaggagcgcctgaccaaagtggttttgccctgcttcccgcaagaggtggcacccacggctggaacgcaggagtcagacccacagtccccagctctggacgcccgcagcggggcctcgaagaggttcagggcggtgcccgcggcgctcgggccgggtctcccggggcgtggggcggggggcggggttgggcggcggccggggctcctccctcttctgccccgggctcccctgctcttaacccgcgcgcgggggcgcccaggccactgggctccgcggagccagcgagaggtctgcgcggagtctgagcggcgctcgtcccgtcccaaggccgacgccagcacgccgtcatggcccccgcagcggcgacggggggcagcaccctgcccagtggcttctcggtcttcaccaccttgcccgacttgctcttcatctttgagtttgtgagtggctcctggccggggaagggacggggtgggctgagccgtgcgctctctcgggcgcccagcacagctgtcggacgggatccgctagctgcgcaggttctgggagcatcggggcagcaggcgcagggcggggactaagccagggaagtcccctcccacctccggtcctttgtgcccttctagaccaacagaatgaggggaacagtctacaggactatggaggaaaaactgggttcccaactggggtcagatgtaggcagcggggcagggggggacggctcttggttcgctggtcccaaagctgcgcgcggggcccacttgacgcgcgcagcgccaccgaagctcccgccgcgctttgcgcggttgggtagaagtgcgcagcttttacaagggagaaggtttcgttaaaaaagaaaaaaaaatcagcaagagaaacattagtattaccaaccgagatttggagatgagagggagctgaatccggtttattttcttctggccttttaaagtttctggcgagggaacgtatttgcgaccaattcgat

SEQ ID NO: 33 - DNA, 1650 bp, Homo sapiens
agtgttctcaagggaccgctctacgacgccatgctttgcatactaaagagcatggatttactagttacctgtgtaatcttcagtaaacgatttaacttctctgtgcttcagttatctcatctataaaatagagatcagatgaactaatcccagtaaaatgaataccattaagtgacactgattatcagttacttcacttgcggaagagtggagggcatgactaggaatggggtgggaggagggagtcaaagaagtcttagctgaattttttttttttttttttttttttttgagacgaagtctcgctctgtcgaccaggctggagtgcagtggtgcgatctcggctcactgcaagctccgcctccccggttcacgccattctcctgcctcagcctcccgagtagctgggactacaagcacccgccaccacgcctggctaatttttgtatttttagtagagacggggtttcaccgtgttagtcaggatggtctcgatctcccgacctcatgatccacccgcctcagcctcccaaagtgctgggattacaggcgtgagcaaccgagcccggccccagctgtattttatacattaaaaaagaaaagtatttaggtattcatacgtgggccgagttttctcctctctcatacaagcacattacacgcgaagccagattagttcatgaatgtgctactgcacggggtggctaagaaatcctgcttgcaaaccgctttgggtcctgcgtggagaatggtttcgagtgagagccgaaccctaaatccgtcttccttatgtggagctcaacgcgactctcaggtattcaggaagaataccttttgctcagcacctgcggagtggtggccacagcgaggcgctcgggagaggcgcctggaggccggcagtgggggcgcgccgcctgagcaggggtgcggggcggggagaaggccggcccacgtggaccgcggggccaggcagggacaggagcagccgggcggcccggccaatcagcgcgttccgcggcgggcccggcccctcctggtcagcgcgctagctgggctcggctccgcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtgagcgtcctcgggctccggtgcggcgatcagtacctagttccggacgcgccggtccgacttggatgccggctctagtcgagtaagaagggttggaagggataaggaggggcgagaggatggggtgggggtggatttggaccctgtatttaggtgctgtctcgtgggcagccgctgcctctcggctggtaccgagttaactcagctcggtgcagctcccctcatcccggctctctggggcgccggggagagtgcctgtgctgaggtcggcgtgcaacccgaagttggaaggggcactccgaagtaaggatgtgtggctggagagaggcagcggccgcttccagtttcggggttcatgtct

SEQ ID NO: 34 - DNA, 1650 bp, Homo sapiens
gtgctaatctgatgggggcagccttagggagggctctgtccccagcaggaggacccttggtgagcgactctccccgcttgcccccaccccttccagcacctgcaaagggggcttagagcggggctgccccatggggttgagaaattgaaatgggggtgtcctcccccaggtgtctctgtggtccttgaatcctggtgtctgttgaaatgcctcctcctctgggcaccttccaggaccaggccaggtgacaccaccattccacccccgtctctacccctttcctggactttcttccttccctgccctggctggggaagcagcggctgcctgcctgtgttcaccccacaccaccgctggggttgtgagaatcgctccttagcaccctggaggacacctctgtgagtggcagccctggggggtgtcgcctcccttgcctggcccctcaggttcctaagggcgtctgaggaaaagcctcattggaaagcgcccgtgccctcctccctctcccccgcccccccgccccccggcagggaggagggttcggagcgagggttcggagggtccggggtgccctagcaggcccaggccaggtggttgcggggaggaagcctcaggtcgctcccctccggtgccggggtgggcgcgtcgggacccactggcctcccagtcccaggagaggaaggaccagggaaaccagctgtctgcagctgctggggaaattaggggctctcagggttccctttcctccgggaggggaggaaacgggactaggaagctttcctgtttctagagcacggcttggtggggtgggcggctcctaacccggggtccagcccctcctcccccaccccggcgcgcggtccaggtatgtgcgacccctcccagtggccacgcgccttctcacgcccctctcccgtgacgtcatgctcctctcgcgcggcatgatgggagaatcctaatgttttccaacagatgctccaagaacagctttcagattaaagcaattgcaagagcaaagattcttccttttcccttttttcctgggggggtggggggtggggtggggggagggagcgcccccagacattccaggacatcaccccctgccccaagcacgcaataaacactgacaagaaaaagtttttatttcctggttcaacttttttttttttccctggaatatagactgaagaatgggaataaacacgaataaataacaaagcgaggccgcgcacgccgggatgcgcctggctgcagccggcgaggctattgtctccccgccctgaagccagccccggcgtctttctccagctcgtttcctttcccggccttttttttttttttttttttttaattttcttttttaaaaagacgccccctccagccccctcgccggtgaccttggccgcctcggatgctctgattccacgcggctcgctctaacttgcccccgcgccggccgggcccatggccgcgtcggaggacgggagcggctgcctcgtgtcgcggggccgctcgcagagtgaccccagcgtcctcaccgactcctcggccacctcctccgcggacgccggggagaacccaggtaacgggctgggcgggggcggcgggcgggtcggggaggcccgaggcaggtggcgctgagagcag

SEQ ID NO: 35 - DNA, 1700 bp, Homo sapiens
cccgccagctttagggactctggcggcgtctcttagccgggtagatgttcctcctttccctctctcttccaagccgctgccaggccgggggtgaggctgcacctcagtcttcagccccgaaggccgcgcccggggcgggtcgctccccattcagctaagaggaagcagcggccggggggcaggcgaggaacctgtcccggctcacccggcaagactgcgtttcccccaccctcggcgcttctttctgtgaatgtgaatggaactaagcgttcctttctctccctcaatggccggcgaggagaattcaaatgcggtctttgggcgctcggtatgggtctggaccttggttttcagagcctgcgcgtccaaggtcgctagcctttcccgaaggaagtgacagctgcggcggcgcgggaggcccatcctcggtcgtttaaagccacttaaggaataaaagcccccgagagagcaaaacgaccacccgaaacctcggaagcgtcgggggtgccgggcccaacgctcccacgtgtcactgccggggactcgcgaggcgaagcttcccagcgctcaaggcgctcccgcctcgtccccaggtatcaggaacagacacccaccaggtccaccagcgggtcagacgccgccttcgggcaggctgggatcttccgaactgggagcggagagtaaggggctcgaccccgggacgcgagtccggccttctggactccggttcagggtgtgtgtgcgcgcggaggggcttatctgggggccctgggtagcacacgtgttcggtttttttctccccgactccgcacgcctggagcggcaatactgcctgccctagaaggccagcggcgagtgctcgccactagggtcccagggagggtttggaaaactgatgagttaagtgagcgaccccaggggacagagggcgagtcgagagtcggccaatggctgcggtgggcggggagaagacgacgcggggatctgcgtgggccgggtcaattccctaccctcgacctgtcgatgccccgcggccccgcccgccctcttaagcctggctcagccctcagggcccgcccgaagtctaccgagcccgagtggcctaccgagcccgagtggccccgcagcgtccaggaggcgcccgctccgcggtggcgctcttggaggtggtgtcggaggtaggcacgggacaggacgcgcctggggcccggggcggtggtcttccagggccgttggggagggcggcagcactcgctggcgcagttcgttttggatggtcgttctgccctctcgggggctttgaatcccaagttgcagatccctgaggtcggaggaggctaggagaagggcgcctttggaggatcgggaggagacgggccgctgcctgtgtcgtggctgacctcttctcctgaccccgtgttctttaatttctgagtcatgaccctgcttggttttcttattgggctcattgatctcaagacccgccggccctgaaggggcttcattcttcagcctcggtgaacttgctgcctgtctattaaaacgccatcctttcccggcggtgcgggggcgggaggactggcagtcgccgaggctcttcgctcccacctggccagggctcgtccacgcggctcccgaggggctaccccaggccccaatagtcctggtagaatgattggagtttccgaggaacccgggga

SEQ ID NO: 36 - DNA, 1650 bp, Homo sapiens
gggagttcctagaccttatatgtctaaactggggcttcctgacataaaactatgcttaccggccaggaatctgttagaaaactcagagctcagtagaaggaacactggctttggaatgtggaggtctggttttgctcaaagtgtgcagtatgtgaaggagaacaatttactgaccattactctgccttactgattcaaattctgaggtttattgaataatttcttagattgccttccagctctaaatttctcagcaccaaaatgaagtccatttcaatctctctctctctctttccctcccgtacatatacacacactcatacatatatatggtcacaatagaaaggcaggtagatcagaagtctcagttgctgagaaagagggagggagggtgagccagaggtaccttctcccccattgtagagaaaagtgaagttcttttagagccccgttacatcttcaaggctttttatgagataatggaggaaataaagagggctcagtccttctactgtccatatttcattctcaaatctgttattagaggaatgattctgatctccacctaccatacacatgccctgttgcttgttgggccttcctaaaatgttagagtatgatgacagatggagttgtctgggtacatttgtgtgcatttaagggtgatagtgtatttgctctttaagagctgagtgtttgagcctctgtttgtgtgtaattgagtgtgcatgtgtgggagtgaaattgtggaatgtgtatgctcatagcactgagtgaaaataaaagattgtataaatcgtggggcatgtggaattgtgtgtgcctgtgcgtgtgcagtatttttttttttttaagtaagccactttagatcttgtcacctcccctgtcttctgtgattgattttgcgaggctaatggtgcgtaaaagggctggtgagatctgggggcgcctcctagcctgacgtcagagagagagtttaaaacagagggagacggttgagagcacacaagccgctttaggagcgaggttcggagccatcgctgctgcctgctgatccgcgcctagagtttgaccagccactctccagctcggctttcgcggcgccgagatgctgtcctgccgcctccagtgcgcgctggctgcgctgtccatcgtcctggccctgggctgtgtcaccggcgctccctcggaccccagactccgtcagtttctgcagaagtccctggctgctgccgcggggaagcaggtaaggagactccctcgacgtctcccggattctccagccctccctaagccttgctcctgccccattggtttggacgtaagggatgctcagtccttctaaagagttttggtgcttttctgggtccctcagctcccgaagctcttgagaaaactatcaaaggctagaatccccttctaactctttttttcccccatgataagcgcagtcggtcacagttcaggtgagttcttacttggcattcaagaaaattacaaaatctgggtagttgtctgggcacgaagcgacaatggcgtctatccctggtgctgaccctgggaagcgctgacccaggtgctgaaacgcagacctctgaagctgctacctcttagcgtacctcacttccaaacgtcgggactagggcaaag

SEQ ID NO: 37 - DNA, 1650 bp, Homo sapiens
caggagtgaggagagacagggttagcgggggcagtcgaaggagacaacggaaaggcagaaaacagaaaaataacgcaagagagagaaaaagtaaaggaaactggcaacaacgtttcaaatttgccattaaaaaattcaaacctcaataacctgggaacttaagcgctttcatccacctgtgtgtgtgagtgtgtgtgtgtgtgtgttttcggggaggggggtgcggtggtgccggaagtctgattagataaaagcgagaaatacagaggctgccggttacactgagaaattacatttttttgtaaagagagcatagtgcccgtgtgctcccaagatcttaaattataaacaggagggggacaaagcaagagggaaacaaacttcaaaaggagcaaataacaaaagcctcttttgctgctcttgaagggaggggggaagggggaagaaagaaaagaaagttgctgaatcgggacattctggaagtgcctttgctgtgtttactagccccatccccgcctgctgcacccggagacgtctgaagtctctaatcgctcagcgaaaagttggtttcgggaagggcagcgccggggtgcgggagggcaaaggaggaagaggaatgcatatgactgtgagaaggaaaagtaatcgtggcaaaaaaaaaaaaaaaaaaaaaaaaagaaagaaagaaaagaaaaaagaaaaaaaatttccgtgagaagagggaaaaaatttggctaaaaaaaaagttgctactcctggcagccctggtttgtcaaaaggggatgtcaagcgctttacaatacctgggattgatgaggcgggcgggccaatgagctgcgcgcggcgcctcggcgcgccctccgttggcgcggcggctgagggcggggggaatgcgggcgcaccaatgggcgccagcgtcggcagcacgtgacacctcccccctgctcccattcatcaagggggggacggtgtcgtcctttcaattcatttatctgcaggaatgattgctgctatcagtctcgcgctcaccgcccggctgaggaggtgaaagtttctccccaggaagataaaccgcaaaagacaatattgtgcatgatttgcgccttttctttggctttttctttctttcttcacccccccacccacttttttttttttttttttcaaaaagcagagagggaaaaacggagagtgaaggagcgaggaggcgagcgtgagagaaaggagagagagagaaaagaaagggcgaggggctagtggagaagtaaggaggggcgcgctgcgcgaggcggagagagggcgaagcagtcgcggcactggcgctcacattcctctatgctacaaatccaggaggaagtttttttttagggggctgagatgctccatgcctttccccgggcagccttgacgcgcggccctctcggcagagactgagcggcgagaaagtgcgagccgggccggcagaatctgcctggcgggcgctggagcctgcgttactcgcggcccgcagccgtccggctactttgcgtttggcccggccagcgcccgggcgcgccgcgccattgcctgcaggctaggacttcgcgaggtgggtcgactccccctccctcctcctcttcttcctcctcttcctcctcctcttgttcctcc

SEQ ID NO: 38 - DNA, 1650 bp, Homo sapiens
gcgggggggtgggctgcagagttgaaagtgcacctgaggcgaatgggccattaaacacccaatactgagacggtttcggggaggatgcggagggaggagcttcacctagttgagcattcagaactttgggtgggaaagggggtgttgagacattctcaattttctagaaataggatgactagtgtggagtttgggtttattttatgttcatctttgtcatttttcgtctggatcttctggcttgggtcccgttgggagaggtcgctctcttaaacctctctttcccaatacatcgcgctaccccctcacttgggctggaatgaggagggggtcctgaaccgctttttttggcaacaacaatattttaacatgttccagtctaggttttgttgaataattagaaatggaaatatttttttctccctcccccttctgaaaaaaaagggggaaatgtgagctctcttctgattggtgggtgacgattttagtccgcaccaatgagctggcggcctcgtctgtatttatttagtgctctgggggagtttttctctttacgcagcgcctgtagtcacggctggaaccttggctgggggactctgagggataagaggggattcggggggaatccgaactgtacctgttcagagatccgcgaccttgcagccggggctggcggaactctgatagggagatagagggataggacaggcaggcggggaggctccgggtgctggggcgacaggaaaggggtggaggctgggctccgggatccgggtgcccctctgcggtccccttctcgccctccacacacttctctcggggctgagttgggggatcgcggacggtctggagaaatggagacacgagtccaggggcgcggaggggcgggcagcgcgcggagtggtgagactgagccgcgatggaacgcgctggggagacccagcctgttcggctccagggttcggagacatcctgggctgaaggcggcggcgaaccgaagaagccggcatattctgcccttcgcgaatctttttgcggtctgggcttgctgtacataactcaatagccggaagcccttaccccaaaaagcatttgcggagggcgcactcgtcgagaagacggcagccatccagcgatcgccgaagcccgcaccttcccgaagctgctccatccgagccttacccggccctttccccgttttccacggaagattcggccctttcccccgtcctttccgcggtgcccgaggctttggtcacctgtcttccctccgagcgagtcccccgcccccaactttttaacagactttgtatcacagggggaactggagcaggtggggtgtgtgtgttttcggagaagtgagaagaagctaaaatgcaataaaagtttacagaagttgtgtcttccccccaacccccgcccacaccacgaataaccccattcactcaatgcaaagaaaaggatctgagcttaaggccaagtcagaacctggaatatgaatcagtttaggacattttgtggtcatttttgaccgtaatgatccgattctgcaatatgggggtggggaagcccttgagggaggggctggaggcccttagtatcttatgtcctggtgcgggggacccggaggtgaggcgagccgcggtgaat

SEQ ID NO: 39 - DNA, 1650 bp, Homo sapiens
gaccaccgcccccaaccccgggagcctctgggcgccccgcactccgccagccgcccgtgcaccccggcccgggggacgcagcccactgcccctccggccttgtctcttaggggtggcgggggatggaacggtggcgaggggtttgggggaagatgggaaagcactttccagacctgttgcacgcgcgcaacagctgttcaggtgcggtatgtggggggagggggtctgctaggatgggggacagagtggtcgcttatgctgcagggaaaagggggtgccgggagggggagaagagcagtcttggggactgtgtgcaaggttgtgcgcgaacggggtccgcgggaagcactgcctaatggggcagggagaatcccaaagagtgggtgggtggaacggaggggatgggacgacttcgacccgagttcccggggctgggcgcgggcggctggaagaccgggaatagagtactgggaatggctggggaagggggtctcaggggaggccccgcaaagcgggtctttcttgggtcgtcccatcttaggggcttttcccgggcctgtatttgttccccaccgtttaactctttcagctccgaatattcgtcctcgtgcagcgcccccgcccccagctttccagatgggaaggtaaaaagttgccacaagttagagtgagttagctgctggcaaaagtttaaataactaccgagggaggtgagggttcgaccaggaatgggttctaatctgaggacgggtaggaaggaaggagagtgtgcgattcttgcagcccctttgctgtgacattgcgaccagaatttgtcgaatcgggctgttaaaacaccatttctgttgggcatttgctcctgctgactgcggcagtaactttccataattaatgacccgttccattatcggtcgcatcctgaactaaattgcttttaacgagttattcttctttgtcttagctggggggtgggggaacacacctgcaggaagaaacttctggaggaagacagcacgtttggtcttttgaggcaaagttctgagacactccgactctgagtatgatagaagtcagtgcactacagaactttgtctctagaggctgtggtcgccgatgccgccagtgctccagacgccaggccgagtggccctggtggccacgtcgctgcgcctttcccttgggggaggggcaaggccagagggggtccaagtgcagcacgaggaacttgaccaattcccttgaagcgggtgggttaaaccctgtaaatgggaacaaaatccccccaaatctcttcatcttaccctggtggactcctgactgtagaattttttggtttaaagaagaaaaaaataaagctttggacttttcaaggttgcttaacaggtactgaaagactggcctcacttaaactgagccaggagagctgcagatttattaatgggtgtgttagtgtgcagtgccttgcaggaatttaaactctctacccttaaagaaggaaatgtatttgtacaaaagccacagataatggtccttttactcaagcgacctgctggataaatgacagcctgcagggattgagtttggagatctagtctcaattctgcttccgatttagatgcagttcacttttgttttttaaagtaagtttta

SEQ ID NO: 40 - DNA, 1700 bp, Homo sapiens
aaactcatctcatcaaaggtgggttttagaccagtcagggcagggcctggactcgctgggagtggggcttaggccacctaggggtggggcctggaccgcttggggtggagttaccaccagccagggtggggtttagaccagcagagttgggaccttagctagaggagtggggtttatgccatgtgggggcagcccagaccatctgggggtgggtcctagaccacctggggcagagtttagaccagccagggctgggttagaccggcagaggtgggccgtgggctatctgggggcaggacctggaccatccagggccgtgtctggaccattcagggcagggcctggaccacctgggggtggaatttagaccatctgagggggtgcttgagcctgcctgggccaggaccacgcatgatgcagtctggaccattggcggccctacaccacctgggggatggagtttcgaccagccggggttggattagaccagcagaggtgaggcctgggccagctaagagtgggccggagccctctgggttggagcttagaccagcagggaagcgcctagagcatcgggcggggagttgtgttcacttctggcaggacttgggtcagccagatccaggacgggggtctagaccactcggggtggggcctagaccgcctgggggtggcgcctgggcccgctggcgcaggcgggcaggggtcggggccgggggcgggggcggggccgggggtggcctcgaggctcccgtctgaccgtccccgcccaggcctgagcaacatcatcggcgtgatcgtgtacatctccgccaacgcgggcgagccgggcccgaagcgggacgaggagaagaaaaaccactactcgtacggctggtccttctacttcggcgggctgtcgttcatcctggccgaggtgataggcgtgctggccgtcaacatctacatcgagcgcagccgcgaggcgcactgccagtctcgctcggacctgctcaaggccggcgggggcgcgggcggcagtggcgggagcggcccctcggccatcctccgtctgcccagttaccgcttccgctaccgccgccgctcccgctctagctcccgctccagcgagccgtcgccgtcgcgggacgcgtctcccggcggccccgggggcccgggctttgcctccacggacatctccatgtacacgctcagccgcgacccctccaagggcagcgtggccgcggggctggcgggggccggcggcggcggcggcggcgccgtgggggcgttcggcggcgcggccgggggcgccgggggcggcggcggaggcggcggcggggcgggtgccgagcgggaccgcgggggggcgtccggcttcctcacgctgcacaacgccttccccaaggaggcgggcggcggcgtcacggtcacggtcaccgggccgcccgccccgcccgcgcccgcgccacccgcgccctctgcgcccgcccccgggaccctggccaaggaggccgccgcctccaacaccaacacgctcaacaggaaaaccacgcctgtgtaggggcgcggcgggggagccgaggggcgtgtccggggcgcgtgcgcgggcgcgcgtgcatcgaggctgccggggtcgggggcgcccccgctttcccccgtgagcgcgctggagactgctgggcccgccccacgcccaccctccccgcccccctccccct

(19) United States
(12) Patent Application Publication
(10) Pub. No.: US 2021/0172029 A1
(43) Pub. Date: Jun. 10, 2021

(54) URINE DNA METHYLATION MARKERS FOR BLADDER CANCER

(71) Applicant: Stichting VUmc, Amsterdam (NL)

(72) Inventors: Jacobus Adrianus Nieuwenhuijzen, Amsterdam (NL); Renske Daniëla Maria Steenbergen, Amsterdam (NL); Judith Bosschiether, Amsterdam (NL)

(21) Appl. No.: 17/258,302

(22) PCT Filed: Jul. 11, 2019
(86) PCT No.: PCT/NL2019/050432

§ 371 Date: Jan. 6, 2021

(30) Foreign Application Priority Data
Jul. 11, 2018 (EP) 18182984.7

(51) Int. Cl. C12Q 1/6886

(52) U.S. Cl. CPC C12Q 1/6886; C12Q 2600/154; C12Q 2600/112

(57) ABSTRACT

The invention relates to methods for detecting the presence of, or risk at, bladder cancer using novel DNA methylation markers for bladder cancer and to kits of parts useful in such methods.

### FIELD OF THE INVENTION

The invention relates to the field of cancer diagnostics. In particular, the invention relates to methods and means for classifying an individual as having bladder cancer or recurrent bladder cancer or being at risk of developing bladder cancer.

### BACKGROUND OF THE INVENTION

Bladder cancer (BC) is one of the most common cancers in the Western world. In the majority of cases, it presents as non-muscle-invasive bladder cancer (NMIBC). In these tumours, recurrence rates are high with 5-year probabilities ranging from 31 to 78%. Current gold standard for the diagnosis of (recurrent) BC is flexible cystoscopy, which detects most cancers, but is an invasive procedure. Furthermore, cystoscopy is operator-dependent and places a significant burden on health care economics. Urine cytology is non-invasive, but has a low sensitivity for low grade tumors. Additionally, the diagnostic value of cytology depends on the expertise of the pathologist.

Tumor suppressor gene silencing by promoter methylation is an established phenomenon in oncogenesis. Previous studies, such as Chan et al. (Clin Cancer Res, 2002) have analyzed aberrant DNA promoter methylation in urine for their diagnostic potential in BC patients. Several urine methylation markers of protein coding genes have been identified with sensitivities ranging from 52 to 100% and specificities from 0 to 100%. However, none of the methylation panels have yet been implemented in clinical diagnostics to reduce the number of cystoscopies. This is mostly due to poor diagnostic accuracy for low-stage and low-grade tumors (Chou et al. 2015, Ann Intern Med).

There exists a need in the art for a non-invasive, objective test with high diagnostic accuracy for all stages and grades of bladder cancer. Such a test minimizes the burden for patients and reduces health care costs. Such a test could further decrease the number of cystoscopies and thereby improve the quality of life of BC patients.

### SUMMARY OF THE INVENTION

It is an object of the present invention to provide novel and improved combinations of hypermethylation markers that can be used for diagnosis and prognosis of (recurrent) bladder cancer. These novel markers allow for diagnosis or prognosis with a high sensitivity and specificity.

Accordingly in one aspect, the invention provides a method for classifying an individual as having bladder cancer or recurrent bladder cancer or being at risk of developing bladder cancer, the method comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935 and classifying said individual based on said DNA methylation.

In a further aspect, the invention provides a method for typing a urine sample from an individual, the method comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935 and typing said urine sample on the basis of DNA methylation.

In a further aspect the invention provides a method comprising determining DNA methylation of at least a first gene or a promoter region thereof and a second gene or a promoter region thereof in a urine sample from an individual, wherein said genes are selected from the group consisting of GHSR, MAL, FAM19A4, PHACTR3, PRDM14, SST, ZIC1, miR-129, miR-148 and miR-935.

In a further aspect, the invention provides a method for determining a treatment strategy for an individual, comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935, and determining a treatment strategy for said individual if determined DNA methylation indicates that said individual is having bladder cancer or recurrent bladder cancer or at risk of having bladder cancer.

In a further aspect, the invention provides a method of treatment of an individual in need thereof, comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935, and providing said individual with bladder cancer treatment if the determined DNA methylation indicates that said individual is having bladder cancer or recurrent bladder cancer or is at risk of having bladder cancer.

In a further aspect, the invention provides a method for monitoring the treatment and/or progression of bladder cancer in an individual, the method comprising determining DNA methylation with a method according to the invention at a first time point and at a second time point

In a further aspect, the invention provides a kit of parts comprising means for the detection of DNA methylation in at least a first gene or a promoter region thereof and a second gene or a promoter region thereof, wherein said genes are selected from the group consisting of GHSR, MAL, FAM19A4, PHACTR3, PRDM14, SST, ZIC1, miR-129, miR-148 and miR-935.

In a further aspect, the invention provides a use of kit of parts according to the invention for classifying an individual as having bladder cancer or recurrent bladder cancer or being at risk of developing bladder cancer.

In a further aspect, the invention provides a use of a urine sample comprising a preservative, preferably ethylenediaminetetraacetic acid (EDTA), and optionally an antibiotic for analysis of DNA methylation of one or more genes or a promoter region thereof.

### DETAILED DESCRIPTION

The present inventors have identified ten genes of which the promoter regions were significantly hypermethylated in urine sample of bladder cancer patients using quantitative methylation specific polymerase chain reaction. It was further found that the use of a combination of at least two of these markers enables prediction of the occurrence of bladder cancer with particularly high specificity and sensitivity. In addition, contrary to know methods that use genetic markers for diagnosing bladder cancer, the methods of the present invention allow the diagnosis of any stage or grade of bladder cancer. This has the advantage that the same markers and thus the same test can be used for diagnosis or prognosis for all grades and stages of bladder cancer. It is no longer necessary to use different genetic markers or combinations of genetic marker to include all grades and stages of bladder cancer.

In a first aspect, the invention therefore provides a method for classifying an individual as having bladder cancer or recurrent bladder cancer or being at risk of developing bladder cancer, the method comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935 and classifying said individual based on said DNA methylation. Table 1 shows the genes, their full name and RefSeq ID. The promoter regions of the genes are shown in FIG. 9.

The term “bladder cancer” as used herein refers to any type of cancer of the bladder. Bladder cancers can be staged according to the TNM system. In this system, letters are assigned numbers to describe the cancer:
Ta: A non-invasive papillary tumour.
Tis: A carcinoma in-situ.
T1: Tumor has invaded subepithelial connective tissue, but not the muscular bladder wall.
T2. Tumor that has invaded the muscular bladder wall but is still confined to the bladder.
T3. Tumor has spread through the bladder wall to perivesical tissue.
T4. Tumor has invaded nearby structures such as the pelvic wall, seminal vesicles or uterus.
N0-N3. Indicates the absence or presence of regional lymph node metastasis.
M0-M1 Indicates the absence or presence of distant metastasis Bladder cancer grading refers to how the cancer cells look under the microscope as compared with normal cells (differentiation of tumor cells).
Grade 1: well differentiated
Grade 2: moderately differentiated
Grade 3: poorly differentiated.
   In addition, bladder cancers cells are also referred to as low grade and high grade. The recurrence rates of bladder cancer after primary treatment are high despite intravesical treatments such as transurethral resection and/or adjuvant intravesical chemotherapeutical and immunotherapeutic instillations.

The bladder cancer that is diagnosed, determined, typed, etc. with any one of the methods of the invention can be non-muscle invasive bladder cancer (NMIBC) and muscle invasive bladder cancer (M1BC). NMIBC refers to stage Ta, Tis and T1 tumors. MIBC encompasses stage T2, T3 and T4 tumors. In a preferred embodiment, the bladder cancer is NMIBC.

The bladder cancer that is diagnosed, determined, typed, etc. with the methods of the invention can further be a primary tumor or recurrent bladder cancer. In a preferred embodiment the bladder cancer is a primary tumor.

The term “recurrent bladder cancer” as used herein refers to forms of bladder cancer that have reoccurred after an intervention, typically surgical intervention, to remove existing bladder cancer.

Currently known urine-based tests have not been able to replace cystoscopy. This is mostly due to poor diagnostic accuracy for low-stage and low-grade tumors. The present inventors have found that the diagnostic performance of a combination of two hypermethylation markers as described herein, in particular the combination of GHSR and MAL, surprisingly did not depend on grade or T-stage. Grade 1-2 tumors were detected with a sensitivity of 93% while grade 3 tumors were detected with a sensitivity of 94% Likewise, Ta-T1 tumors were detected with a sensitivity of 95% and stage T2-T4 tumors with a sensitivity of 92%. Positive DNA methylation of at least one of these two markers (‘believe-the-positive’) resulted in a particularly high sensitivity of 92% (95% confidence interval (CI): 86-99) and specificity of 85% (95% CI: 76-94).

A method of the invention comprises determining DNA methylation of at least a first gene or a promoter region thereof and a second gene or a promoter region thereof. In particular, DNA methylation of genomic DNA is determined.

Preferably hypermethylation of these genes and/or promoter regions thereof indicates the individual is likely to have bladder cancer or recurrent bladder cancer or at risk of developing bladder cancer in the future. The term “hypermethylation” as used herein refers to any methylation of cytosine at a position that is normally unmethylated in the relevant gene sequences (e.g. the GHSR and MAL promoter regions).

Preferably, DNA methylation or hypermethylation of the promoter regions of the at least two genes is determined. In a further preferred embodiment of the invention the DNA methylation or hypermethylation is detected in the CpG rich sequences in the promoter regions of the at least two genes. FIG. 9 shows the promoter regions and CpG rich sequences of the genes.

In accordance with the invention DNA methylation of at least two genes and/or a promoter region thereof is determined, wherein the at least two genes are selected from the group consisting of growth hormone secretagogue receptor (GHSR), myelin and lymphocyte protein (MAL), family with sequence similarity 19 member A4 (FAM19A4), phosphatase and actin regulator 3 (PHACTR3), PR domain-containing protein 14 (PRDM14), somatostatin (SST), Zinc finger of the cerebellum protein 1 (ZIC1), miR-129, miR-148, miR-935.

In a preferred embodiment, a method of the invention comprises determining DNA methylation of GHSR or a promoter region thereof or MAL, or a promoter region thereof and a second gene selected from the group consisting of GHSR, MAL, FAM19A4, PHACTR3, PRDM14, SST, ZIC1, miR-129, miR-148 and miR-935, or a promoter region thereof. In a further preferred embodiment, a method of the invention comprises determining DNA methylation of GHSR or a promoter region thereof and a second gene selected from the group consisting of MAL, FAM19A4, PHACTR3, PRDM14, SST, ZIC1, miR-129, miR-148 and miR-935, or a promoter region thereof. As shown in the examples, all these combinations have a particularly high sensitivity of over 80%. It is preferred that the methods of the invention comprises determining DNA methylation of the promoter regions of these two genes.

In a particularly preferred embodiment, a method of the invention comprises determining DNA methylation of GHSR or a promoter region thereof and of MAL or a promoter region thereof. Positive DNA methylation of at least one of these two markers resulted in a particularly high sensitivity of 92% (95% confidence interval (CI): 86-99) and specificity of 85% (95% CI: 76-94). In addition, the diagnostic performance of the combination of GHSR and MAL does not depend on grade or T-stage. Surprisingly, sensitivity for all tumour grades and both primary tumors and recurrent cancer are higher than could be achieved with any individual marker. Grade 1-2 tumors were detected with a sensitivity of 93% while grade 3 tumors were detected with a sensitivity of 94%. Ta-T1 tumors were detected with a sensitivity of 95% and stage T2-T4 tumors with a sensitivity of 92%. It is preferred that the methods of the invention comprises determining DNA methylation of the promoter regions of these two genes.

In a further preferred embodiment, a method of the invention comprises determining DNA methylation of SST or a promoter region thereof and of MAL or a promoter region thereof. Positive DNA methylation of at least one of these two markers resulted in a particularly high sensitivity of 92% and specificity of 79%. It is preferred that the methods of the invention comprises determining DNA methylation of the promoter regions of these two genes.

In a further preferred embodiment, a method of the invention comprises determining DNA methylation of GHSR or a promoter region thereof and of SST or a promoter region thereof. Positive DNA methylation of at least one of these two markers resulted in a particularly high sensitivity of 97% and specificity of 79%. It is preferred that the methods of the invention comprises determining DNA methylation of the promoter regions of these two genes.

In a further preferred embodiment, a method of the invention comprises determining DNA methylation of GHSR or a promoter region thereof and of miR129 or a promoter region thereof. Positive DNA methylation of at least one of these two markers resulted in a particularly high sensitivity of 87% and specificity of 88%. It is preferred that the methods of the invention comprises determining DNA methylation of the promoter regions of these two genes.

In a further preferred embodiment, a method of the invention comprises determining DNA methylation of GHSR or a promoter region thereof and of miR935 or a promoter region thereof. Positive DNA methylation of at least one of these two markers resulted in a particularly high sensitivity of 87% and specificity of 87%. It is preferred that the methods of the invention comprises determining DNA methylation of the promoter regions of these two genes.

In a further preferred embodiment, a method of the invention comprises determining DNA methylation of GHSR or a promoter region thereof and of miR148 or a promoter region thereof. Positive DNA methylation of at least one of these two markers resulted in a particularly high sensitivity of 87% and specificity of 71%. It is preferred that the methods of the invention comprises determining DNA methylation of the promoter regions of these two genes.

In a further preferred embodiment, a method of the invention comprises determining DNA methylation of GHSR or a promoter region thereof and of FAM19A4 or a promoter region thereof. Positive DNA methylation of at least one of these two markers resulted in a particularly high sensitivity of 86% and specificity of 88%. It is preferred that the methods of the invention comprises determining DNA methylation of the promoter regions of these two genes.

In a further preferred embodiment, a method of the invention comprises determining DNA methylation of GHSR or a promoter region thereof and of PHACTR3 or a promoter region thereof. Positive DNA methylation of at least one of these two markers resulted in a particularly high sensitivity of 86% and specificity of 82%. It is preferred that the methods of the invention comprises determining DNA methylation of the promoter regions of these two genes.

In a further preferred embodiment, a method of the invention comprises determining DNA methylation of GHSR or a promoter region thereof and of PRDM14 or a promoter region thereof. Positive DNA methylation of at least one of these two markers resulted in a particularly high sensitivity of 86% and specificity of 85%. It is preferred that the methods of the invention comprises determining DNA methylation of the promoter regions of these two genes.

In a further preferred embodiment, a method of the invention comprises determining DNA methylation of PRDM14 or a promoter region thereof and of SST or a promoter region thereof. Positive DNA methylation of at least one of these two markers resulted in a particularly high sensitivity of 86% and specificity of 78%. It is preferred that the methods of the invention comprises determining DNA methylation of the promoter regions of these two genes.

In further preferred embodiments, a method of the invention comprises determining DNA methylation of:
GHSR or a promoter region thereof and ZIC1 or a promoter region thereof, positive DNA methylation of at least one of these two markers having a high average sensitivity of 84% and specificity of 84%, or
MAL or a promoter region thereof and FAM19A4 or a promoter region thereof, positive DNA methylation of at least one of these two markers having a high average sensitivity of 76% and specificity of 92%, or
MAL or a promoter region thereof and PHACTR3 or a promoter region thereof, positive DNA methylation of at least one of these two markers having a high average sensitivity of 78% and specificity of 86%, or
MAL or a promoter region thereof and PRDM14 or a promoter region thereof, positive DNA methylation of at least one of these two markers having a high average sensitivity of 83% and specificity of 88%, or
MAL or a promoter region thereof and ZIC1 or a promoter region thereof, positive DNA methylation of at least one of these two markers having a high average sensitivity of 81% and specificity of 88%, or
MAL or a promoter region thereof and miR-129 or a promoter region thereof, positive DNA methylation of at least one of these two markers having a high average sensitivity of 73% and specificity of 92%, or
MAL or a promoter region thereof and miR-148 or a promoter region thereof, positive DNA methylation of at least one of these two markers having a high average sensitivity of 80% and specificity of 74%, or
MAL or a promoter region thereof and miR-935 or a promoter region thereof, positive DNA methylation of at least one of these two markers having a high average sensitivity of 83% and specificity of 88%, or
FAM19A4 or a promoter region thereof and PHACTR3 or a promoter region thereof, or
FAM19A4 or a promoter region thereof and PRDM14 or a promoter region thereof, positive DNA methylation of at least one of these two markers having a high average sensitivity of 75% and specificity of 90%, or
FAM19A4 or a promoter region thereof and SST or a promoter region thereof, positive DNA methylation of at least one of these two markers having a high average sensitivity of 82% and specificity of 81%, or
FAM19A4 or a promoter region thereof and ZIC1 or a promoter region thereof, positive DNA methylation of at least one of these two markers having a high average sensitivity of 75% and specificity of 90%, or
PHACTR3 or a promoter region thereof and PRDM14 or a promoter region thereof, positive DNA methylation of at least one of these two markers having a high average sensitivity of 79% and specificity of 83%, or
PHACTR3 or a promoter region thereof and SST or a promoter region thereof, positive DNA methylation of at least one of these two markers having a high average sensitivity of 82% and specificity of 75%, or
PHACTR3 or a promoter region thereof and ZIC1 or a promoter region thereof, positive DNA methylation of at least one of these two markers having a high average sensitivity of 76% and specificity of 84%, or
PRDM14 or a promoter region thereof and ZIC1 or a promoter region thereof, positive DNA methylation of at least one of these two markers having a high average sensitivity of 82% and specificity of 88%, or
PRDM14 or a promoter region thereof and miR-129 or a promoter region thereof, positive DNA methylation of at least one of these two markers having a high average sensitivity of 77% and specificity of 90%, or
PRDM14 or a promoter region thereof and miR-148 or a promoter region thereof, positive DNA methylation of at least one of these two markers having a high average sensitivity of 80% and specificity of 72%, or
PRDM14 or a promoter region thereof and miR-935 or a promoter region thereof, positive DNA methylation of at least one of these two markers having a high average sensitivity of 82% and specificity of 85%, or
SST or a promoter region thereof and ZIC1 or a promoter region thereof, positive DNA methylation of at least one of these two markers having a high average sensitivity of 80% and specificity of 77%, or
SST or a promoter region thereof and miR-129 or a promoter region thereof, positive DNA methylation of at least one of these two markers having a high average sensitivity of 82% and specificity of 81%, or
SST or a promoter region thereof and miR-148 or a promoter region thereof, positive DNA methylation of at least one of these two markers having a high average sensitivity of 78% and specificity of 65%, or
SST or a promoter region thereof and miR-935 or a promoter region thereof, positive DNA methylation of at least one of these two markers having a high average sensitivity of 81% and specificity of 80%, or
ZIC1 or a promoter region thereof and miR-129 or a promoter region thereof, positive DNA methylation of at least one of these two markers having a high average sensitivity of 74% and specificity of 90%, or
ZIC1 or a promoter region thereof and miR-148 or a promoter region thereof, positive DNA methylation of at least one of these two markers having a high average sensitivity of 77% and specificity of 74%, or
ZIC1 or a promoter region thereof and miR-935 or a promoter region, positive DNA methylation of at least one of these two markers having a high average sensitivity of 77% and specificity of 86%, or thereof. It is preferred that the methods of the invention comprises determining DNA methylation of the promoter regions of genes indicated above.

In further embodiments, a method of the invention comprises determining DNA methylation of:
FAM19A4 or a promoter region thereof and miR-129 or a promoter region thereof, or
FAM19A4 or a promoter region thereof and miR-148 or a promoter region thereof, or
FAM19A4 or a promoter region thereof and miR-935 or a promoter region thereof, or
PHACTR3 or a promoter region thereof and miR-129 or a promoter region thereof, or
PHACTR3 or a promoter region thereof and miR-148 or a promoter region thereof, or
PHACTR3 or a promoter region thereof and miR-935 or a promoter region thereof, or
miR-129 or a promoter region thereof and miR-148 or a promoter region thereof, or
miR-129 or a promoter region thereof and miR-935 or a promoter region thereof, or
miR-148 or a promoter region thereof and miR-935 or a promoter region thereof.
   It is preferred that the methods of the invention comprises determining DNA methylation of the promoter regions of genes indicated above.

In a further, preferred embodiment, a method of the invention comprises determining DNA methylation or hypermethylation of at least three genes, or a promoter region thereof, wherein the genes are selected from the group consisting of GHSR, MAL, FAM19A4, PHACTR3, PRDM14, SST, ZIC1, miR-129, miR-148 and miR-935, more preferably of at least four genes, more preferably of at least five genes, more preferably of at least six genes, more preferably of at least seven genes, more preferably of at least eight genes, more preferably of at least nine genes, or a promoter region thereof, wherein the genes are selected from the group consisting of GHSR, MAL, FAM19A4, PHACTR3, PRDM14, SST, ZIC1, miR-129, miR-148 and miR-935. It is further preferred that DNA methylation of hypermethylation of at least GHSR or a promoter region thereof or MAL or a promoter region thereof is determined, most preferably of at least GHSR or a promoter region thereof and MAL or a promoter region. In a particularly preferred embodiment, DNA methylation or hypermethylation of at least a promoter region of GHSR and a promoter region of MAL are determined.

TABLE 1
Genes, RefSeq ID and full name

| Gene | RefSeq ID | Full name |
|---|---|---|
| GHSR | NM_198407 | growth hormone secretagogue receptor |
| MAL | NM_002371 | myelin and lymphocyte protein |
| FAM19A4 | NM_182522 | family with sequence similarity 19 |
| | | member A4 |
| PHACTR3 | NM_080672 | phosphatase and actin regulator 3 |
| PRDM14 | NM_024504 | PR domain-containing protein 14 |
| SST | NM_001048 | somatostatin |
| ZIC1 | NM_003412 | Zinc finger of the cerebellum protein 1 |
| miR-129 | NR_029697 | |
| miR-148 | NR_029597 | |
| miR-935 | NR_030632 | |

It is preferred that DNA is isolated from the urine sample. More preferably genomic DNA is isolated. Method for isolation of DNA or genomic DNA from urine are well known in the art. As an example the Quick-DNA™ Urine Kit (Zyme Research, Orange, Calif., USA) can be used.

Methods for determining DNA methylation or hypermethylation are well known in the art. For instance, the following methods are suitable and well known to a skilled person:
Methylation-Specific PCR (MSP) or quantitative Methylation-Specific PCR (qMSP), which is based on a chemical reaction of sodium bisulfite with DNA that converts unmethylated cytosines of CpG dinucleotides to uracil or UpG, followed by traditional PCR. However, methylated cytosines will not be converted in this process, and primers are designed to overlap the CpG site of interest, which allows one to determine methylation status as methylated or unmethylated. As an example the EZ DNA Methylation™ kit (Zymo Research, Orange, Calif., USA) can be used to convert isolated DNA.
Whole genome bisulfite sequencing, also known as BS-Seq, which is a high-throughput genome-wide analysis of DNA methylation. It is based on aforementioned sodium bisulfite conversion of genomic DNA, which is then sequenced on a Next-generation sequencing platform. The sequences obtained are then re-aligned to the reference genome to determine methylation states of CpG dinucleotides based on mismatches resulting from the conversion of unmethylated cytosines into uracil.
The HELP assay, which is based on restriction enzymes' differential ability to recognize and cleave methylated and unmethylated CpG DNA sites.
ChIP-on-chip assays, which is based on the ability of commercially prepared antibodies to bind to DNA methylation-associated proteins like MeCP2.
Restriction landmark genomic scanning, a complicated and now rarely-used assay based upon restriction enzymes' differential recognition of methylated and unmethylated CpG sites; the assay is similar in concept to the HELP assay.
Methylated DNA immunoprecipitation (MeDIP), analogous to chromatin immunoprecipitation, immunoprecipitation is used to isolate methylated DNA fragments for input into DNA detection methods such as DNA microarrays (MeDIP-chip) or DNA sequencing (MeDIP-seq).
Pyrosequencing of bisulfite treated DNA. This is sequencing of an amplicon made by a normal forward primer but a biotenylated reverse primer to PCR the gene of choice. The Pyrosequencer then analyses the sample by denaturing the DNA and adding one nucleotide at a time to the mix according to a sequence given by the user. If there is a mismatch, it is recorded and the percentage of DNA for which the mismatch is present is noted. This gives the user a percentage methylation per CpG island.
Molecular break light assay for DNA adenine methyltransferase activity—an assay that relies on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase.
Methyl Sensitive Southern Blotting is similar to the HELP assay, although uses Southern blotting techniques to probe gene-specific differences in methylation using restriction digests. This technique is used to evaluate local methylation near the binding site for the probe.
Methylated DNA sequencing, such a genome wide sequencing of DNA digested by methylation sensitive restriction enzymes.
DNA methylation analysis using nanotechnology or lab-on-a-chip analysis.

In one preferred embodiment, a method of the invention wherein DNA methylation is determined comprises isolating DNA, preferably genomic DNA, treating said isolated DNA with bisulphite and performing quantitative methylation-specific PCR (qMSP).

In the methods of the invention, DNA methylation or hypermethylation is determined in a urine sample. In one preferred embodiment, the urine sample is a full urine sample. “Full urine sample” as used herein, means that all components present in urine are present in the sample, nor purification or isolation of components has occurred, although components may have been added to the sample. In another preferred embodiment, the urine sample comprises cells isolated from urine. As used herein a “sample comprising cells isolated from urine” means that the sample does not contain cell free nucleic acid, in particular DNA, from urine. As an alternative, cell free DNA or total DNA from urine can be used. Total DNA from urine refers to the total of both cellular DNA and cell free DNA.

Storage of urine can affect the quality of DNA, and as a result thereof the determination of DNA methylation. In one embodiment, fresh urine samples are therefore used in the methods of the invention, such as within one day of collection of the sample. However, it is also possible to use urine sample that have been stored at room temperature, or that have been cooled or frozen, e.g. at 4° C. or at −20° C. As shown in Examples 2, the present inventors have found that adding a preservative such as ethylenediaminetetraacetic acid (EDTA) yielded a better DNA yield when the urine sample is stored at room temperature during a period of 7 days. In addition, it was found that the addition of penicillin/streptomycin did not negatively influence the methylated DNA analysis. Hence, such antibiotic can be used in urine samples to prevent bacterial contamination. Therefore, the urine sample used in the methods of the invention are preferably treated with a preservative and/or an antibiotic. Other DNA preservations means and methods are also possible such as, for instance, commercially offered by Zymo research (urine conditioning buffer). The preservative is preferably EDTA. The antibiotic is preferably penicillin, streptomycin or a combination thereof. In a preferred embodiment, the urine sample is stored for at least one week prior to determining DNA methylation or hypermethylation. The sample is preferably stored at temperatures of above 4° C., more preferably at room temperature for at least part of said at least one week, such as for half a day, one day, two days or three days. After that, the urine sample can for instance be stored at 4° C., at −20° C. or at −80° C. In one embodiment, the urine sample is stored for at least one week at room temperature prior to determining DNA methylation. As used herein “room temperature” is defined as a temperature in the range of about 16° C. to about 25° C.

The invention also provides a use of a urine sample comprising a preservative for analysis of DNA methylation of one or more genes or a promoter region thereof. The preservative is preferably EDTA, or another chelating agent. Optionally, the urine sample further comprises at least one antibiotic. The at least one antibiotic is preferably penicillin, streptomycin or a combination thereof. In a preferred embodiment, the urine sample is stored for at least one day prior to performing said analysis, such as 1 day, 2 days, 3 days, 4 days, 5 days, 6 days or 7 days, more preferably at least 2 days, more preferably at least three days, more preferably at least four days, such as about 5 days, about 6 days or about 7 days. In one preferred embodiment, the urine sample is stored for at least one week prior to performing the DNA methylation analysis. In a further preferred embodiment, the urine sample is stored for between 1 day and 8 days prior to performing the DNA methylation analysis. The sample is preferably stored at temperatures of above 4° C., more preferably at room temperature for at least part of said at least one day, such as for half a day, one day, two days or three days. After that, the urine sample can for instance be stored at 4° C., at −20° C. or at −80° C. In one embodiment, the urine sample is stored for at least one week at room temperature prior to determining DNA methylation.

In one embodiment, the invention provides a use of a urine sample comprising a preservative for analysis of DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935, preferably at least GHSR and MAL or a promoter region of these genes, more preferably in a promoter region of GHSR and a promoter region of MAL.

Preferably, DNA methylation of said at least two genes and/or promoter regions thereof is compared with a reference and said individual is classified based on said comparison. DNA methylation or hypermethylation of the at least two genes or promoter region thereof is preferably compared with at least a first and a second reference value. Preferably, the reference values are DNA methylation of the same genes or promoter region thereof determined in a sample that is not obtained from an individual suffering from bladder cancer or is at risk of developing bladder cancer. In one embodiment the individual is a healthy individual. As used herein a “healthy individual” is an individual not suffering from bladder cancer, preferably not suffering from any cancer. Such a healthy individual can in one embodiment be a benign hematuria control. Alternatively, the reference values are DNA methylation of the same genes or promoter region thereof determined in a sample obtained from an individual suffering from bladder cancer. Suitable samples for determining reference values include a urine sample from an individual and a pooled urine sample from multiple individuals, wherein the individuals are either healthy individuals or individuals suffering from bladder cancer. In addition, a reference value can be the average of DNA methylation determinations in multiple urine samples from individuals or multiple pooled urine samples from individuals, wherein the individuals are either healthy individuals or individuals suffering from bladder cancer.

If the first and second reference values are based on DNA methylation of the relevant genes or promoter region in one or more urine sample from healthy individuals, DNA methylation of said at least two genes in said sample from said individual that are higher than said reference values indicate that said individual is suffering from bladder cancer or is at risk of developing bladder cancer. If the first and second reference values are based on DNA methylation of the relevant genes or promoter region in one or more urine samples from individuals suffering from bladder cancer, DNA methylation of said at least two genes in said sample from said individual that are similar to or higher than said reference values indicate that said individual is suffering from bladder cancer or is at risk of developing bladder cancer.

Whether or not DNA methylation is higher than or similar to a reference value can be determined using statistical methods that are appropriate and well-known in the art, generally with a probability value of less than five percent chance of the change being due to random variation. It is well within the ability of a skilled person to determine the amount of increase or similarity that is considered significant. Preferably, “higher than” is at least 20, at least 40, or at least 50% higher than the reference value. Preferably, “similar to” is at most 20% difference, more preferably at most 10% difference between DNA methylation determined and the reference value(s).

Also provided is a method for typing a urine sample from an individual, the method comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935 and typing said urine sample on the basis of DNA methylation. Typing preferably comprises determining methylation of DNA, preferably genomic DNA, in the sample. Preferably, typing said urine sample on the basis of the DNA methylation comprises determining whether or not DNA hypermethylation is present in the sample, preferably of the promoter regions of the at least two genes. In a particularly preferred embodiment, DNA methylation of the promoter regions of at least GHSR and MAL is determined. Other preferred combination of genes or promoter regions thereof are indicated herein above.

Also provided is a method comprising determining DNA methylation of at least a first gene or a promoter region thereof and a second gene or a promoter region thereof in a urine sample from an individual, wherein said genes are selected from the group consisting of GHSR, MAL, FAM19A4, PHACTR3, PRDM14, SST, ZIC1, miR-129, miR-148 and miR-935. Preferably, such method comprises determining whether or not DNA hypermethylation is present in the sample, preferably of the promoter regions of the at least two genes. In a particularly preferred embodiment, DNA methylation of the promoter regions of at least GHSR and MAL is determined. Other preferred combination of genes or promoter regions thereof are indicated herein above. A value for the sensitivity and/or specificity of a certain combination can be found in a table 9 and table 10. Combinations can be ranked on the basis of the sum of the sensitivity and specificity scores indicated in tables 9 and 10.

The methods disclosed herein classify an individual as having or being at risk of developing bladder cancer. Preferably, the methods predict the likelihood that an individual is either suffering from or not suffering from bladder cancer. Depending on the stage and grade of the bladder cancer and other factors, such as age of the patient and other health conditions, treatment options can include surgery, intravesical therapy, chemotherapy, radiation therapy and immunotherapy, or a combination of one or more of said treatment options. Accordingly, also provided is a method for determining a treatment schedule for an individual, comprising determining using a method according to the invention as disclosed herein, whether an individual has or is at risk of developing bladder cancer. If it is determined that the individual has or is at risk of developing bladder cancer, it can be determined if and how the individual can be treated. Preferably, such treatment comprises surgery, intravesical therapy, chemotherapy, radiation therapy, immunotherapy or a combination thereof.

Provided is therefore, a method for determining a treatment strategy for an individual, comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935, and determining a treatment strategy for said individual if the determined DNA methylation indicates that said individual is having bladder cancer or recurrent bladder cancer or at risk of having bladder cancer.

Also provided is a method of treatment of an individual in need thereof, comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935, and providing said individual with bladder cancer treatment if the determined DNA methylation indicates that said individual is having bladder cancer or recurrent bladder cancer or at risk of having bladder cancer.

Optionally, a method of the invention for determining a treatment strategy or for treatment of an individual comprises determining the grade or stage of the bladder cancer. Suitable methods for determining the grade and/or stage of bladder cancer include obtaining and analyzing one or more biopsies and imaging of the bladder cancer, e.g. using CT, MRI, x-rays, PET scan, etc. A physician or other health care professional can readily determine a suitable treatment option, such as surgery, intravesical therapy, chemotherapy, radiation therapy and immunotherapy, or a combination of one or more of said treatment options.

As used herein, the terms “treatment,” “treat,” and “treating” refer to reversing, alleviating, delaying the onset of, or inhibiting the progress of a disease or disorder, or one or more symptoms thereof, in particular bladder cancer as described herein. In some embodiments, treatment may be administered after one or more symptoms have developed. In other embodiments, treatment may be administered in the absence of symptoms, after DNA methylation or hypermethylation indicate that the individual is suffering from or at risk of suffering from cancer but the individual is not yet experiencing symptoms. Treatment may also be continued after symptoms have resolved, for example to prevent or delay their recurrence.

The invention also provides a method for monitoring the treatment and/or progression of bladder cancer in an individual, the method comprising determining DNA methylation with a method according to the invention at a first time point and at a second time point. In this embodiment, said individual has previously been diagnosed with bladder cancer or is classified as having bladder cancer at the first time point. In a preferred embodiment, said individual receives treatment for bladder cancer between the first and second time point. It is further preferred that DNA methylation at the first and second time point are compared. A decrease in DNA methylation between the first and second time point, and preferably after having received treatment for bladder cancer, indicates that the therapy is successful in treating bladder cancer. Similarly, a presence of DNA methylation at the first time point and absence of DNA methylation at the second time point, and preferably after having received treatment for bladder cancer, indicates that the therapy is successful in treating bladder cancer.

Also provided is a method for identifying bladder cancer or recurrent bladder cancer in a urine sample of an individual comprising detecting the presence of DNA methylation of at least a first gene or a promoter region thereof and a second gene or a promoter region thereof in said urine sample, wherein said genes are selected from the group consisting of GHSR, MAL, FAM19A4, PHACTR3, PRDM14, SST, ZIC1, miR-129, miR-148 and miR-935 and identifying bladder cancer or recurrent bladder cancer if DNA methylation of said at least a first and second gene or promoter region is detected. The method may comprise identifying the presence or absence of bladder cancer or recurrent bladder cancer. If DNA methylation of said at least a first and second gene or promoter region is detected, the presence of bladder cancer or recurrent bladder cancer is identified or if DNA methylation of said at least a first and second gene or promoter region is not detected, the absence of bladder and recurrent bladder cancer is identified.

In some embodiments, methods disclosed herein comprise obtaining or providing a urine sample from an individual, preferably an individual suspected of having bladder cancer or recurrent bladder cancer or suspected of being at risk of having bladder cancer or recurrent bladder cancer.

In some embodiments, methods disclosed herein comprise isolating DNA, in particular genomic DNA, from the urine sample.

In some embodiments, methods disclosed herein comprise performing bisulphite treatment of isolated DNA.

In some embodiments, methods disclosed herein comprise amplifying bisulphite-treated DNA.

In some embodiments, methods disclosed herein comprise contacting isolated DNA with means for determining DNA methylation of said at least a first gene or promoter region thereof and a second gene or promoter region thereof. Said first and second genes preferably comprise GHSR and MAL. Preferably said means are for determining DNA methylation of GHSR promoter region and of MAL promoter region. Said means preferably comprise primers for detecting methylated DNA and optionally a probe. The sequence of preferred primers and probes are shown in table 2.

In some embodiments, methods disclosed herein comprise detecting DNA hypermethylation of GHSR promoter region.

In some embodiments, methods disclosed herein comprise detecting DNA hypermethylation of MAL promoter region.

In some embodiments, methods disclosed herein comprise initiating treatment of the individual if bladder cancer or recurrent bladder cancer is identified.

TABLE 2
Primer and probe sequences.

| | Forward primer | Reverse primer | Probe |
|---|---|---|---|
| PRDM14 | TTATTAGCGGGTTAGAC | CGTCCCGAAATCGAACCC | CTTACTCTCCGCTCCCAATTCGAA |
| | GTCGTTT | | AAATCC |
| FAM19A4 | AGTCGGGCGGTTCGGTT | CCAAAAACGACGCGCAACT | CCCAACTAACGCGCTAA |
| | A | A | |
| PHACTR3 | GGTTATTTTGCGAGCGG | CGAATACTCTAATTCCACGC | AACCGCGTCGAAAAACGAAAACG |
| | TTTC | GACT | ACTAC |
| MAL | CGCGTAGTATTAAGTAG | ATCTACAATAAAAAATAAA | CCACTAAACCGACGCTAATTCGAC |
| | AGAGGTTCG | ACCGACCG | GCT |
| GHSR | GTTTGGTTTTTGCGGTTT | CAACCCTACCTCGCATTTAC | CTCGATCCAATTCCATCTCGCACTT |
| | TTATTC | G | CC |
| ZIC | GGGCGGGTTAATGAGTT | TCACGTACTACCGACGCTAA | CGCCGCGCCAACGAAAAAC |
| | GC | CG | |
| SST | GCGTTGGTTGCGTTGTTT | CTACAAAAACTAACGAAAT | CCGATAACACAACCCAAAA |
| | ATC | CTAAAATCCG | |
| miR-129-2 | GCGGAGTGGTGAGATTG | AAAATATACCGACTTCTTCG | CCTAAAACCGAACAAACTAAATCT |
| | AGTC | ATTCG | CCCCAACG |
| miR-148a | GTTGTGATATTGCGATTA | AACGAATCATTAATTATAAA | ACAAATACCCAACAAAAATAATAT |
| | GAATTTGTC | AAATTACTACCG | TTTAACAACCCGATTC |
| miR935 | GAGGTGATAGGCGTGTT | CAACCTTAAACAAATCCGA | GCCTCGCGACTACGCTCGATATAA |
| | GGTC | ACG | ATATTAAC |

In one embodiment, the invention therefore provides a method for identifying bladder cancer or recurrent bladder cancer in a urine sample of an individual comprising:

obtaining or providing a urine sample of said individual;

isolating DNA, in particular genomic DNA, from the urine sample;

contacting isolated DNA with means for determining DNA methylation of at least a first gene or a promoter region thereof and a second gene or a promoter region thereof in said urine sample, wherein said genes are selected from the group consisting of GHSR, MAL, FAM19A4, PHACTR3, PRDM14, SST, ZIC1, miR-129, miR-148 and miR-935;

detecting the presence of DNA methylation of said at least a first gene or promoter region thereof and a second gene or promoter region thereof, and

identifying bladder cancer or recurrent bladder cancer if DNA methylation of said at least a first and second gene or promoter region is detected or identifying the absence of bladder cancer or recurrent bladder cancer if DNA methylation of said at least a first and second gene or promoter region thereof is not detected.

The invention also provides a method for classifying an individual as having bladder cancer or recurrent bladder cancer or being at risk of developing bladder cancer, the method comprising:

obtaining or providing a urine sample of said individual;

isolating DNA, in particular genomic DNA, from the urine sample;

contacting isolated DNA with means for determining DNA methylation of at least a first gene or a promoter region thereof and a second gene or a promoter region thereof in said urine sample, wherein said genes are selected from the group consisting of GHSR, MAL, FAM19A4, PHACTR3, PRDM14, SST, ZIC1, miR-129, miR-148 and miR-935;

determining DNA methylation of said at least a first gene or promoter region thereof and a second gene or promoter region thereof, and

classifying said individual based on said DNA methylation.

The invention also provides a method for typing a urine sample from an individual, the method comprising:

obtaining or providing a urine sample of said individual;

isolating DNA, in particular genomic DNA, from the urine sample;

contacting isolated DNA with means for determining DNA methylation of at least a first gene or a promoter region thereof and a second gene or a promoter region thereof in said urine sample, wherein said genes are selected from the group consisting of GHSR, MAL, FAM19A4, PHACTR3, PRDM14, SST, ZIC1, miR-129, miR-148 and miR-935;

detecting DNA methylation of said at least a first gene or promoter region thereof and a second gene or promoter region thereof; and

typing said urine sample on the basis of DNA methylation.

The invention also provides a method for treatment of an individual in need thereof, comprising:

obtaining or providing a urine sample of said individual;

isolating DNA, in particular genomic DNA, from the urine sample;

contacting isolated DNA with means for determining DNA methylation of at least a first gene or a promoter region thereof and a second gene or a promoter region thereof in said urine sample, wherein said genes are selected from the group consisting of GHSR, MAL, FAM19A4, PHACTR3, PRDM14, SST, ZIC1, miR-129, miR-148 and miR-935;

detecting DNA methylation of said at least a first gene or promoter region thereof and a second gene or promoter region thereof; and

providing said individual with bladder cancer treatment if the detected DNA methylation indicates that said individual is having bladder cancer or recurrent bladder cancer.

The invention also provides a kit of parts comprising means for the detection of DNA methylation in at least a first gene or a promoter region thereof and a second gene or a promoter region thereof, wherein said genes are selected from the group consisting of GHSR, MAL, FAM19A4, PHACTR3, PRDM14, SST, ZIC1, miR-129, miR-148 and miR-935. Such a kit may comprise one or more of the following components: a container for collecting urine, a container filled with preservative and/or one or more antibiotics, and test tubes for analysis. Said means for detection of DNA methylation may comprise primers and optionally a probe suitable for MSP or qMSP of the genes disclosed herein or a promoter region thereof, preferably primers as described herein, and/or methylation-sensitive restriction enzymes. Preferably, said means comprise primers suitable for determining DNA methylation of a GHSR promoter and MAL promoter. Said means for detection of DNA methylation may further comprise means for isolating DNA, preferably genomic DNA, and/or bisulphite for converting isolated DNA.

Also provided is a kit of parts according to the invention for use in a method of classifying an individual as having bladder cancer or recurrent bladder cancer or being at risk of developing bladder cancer according to the invention.

Also provided is a use of a kit of parts according to the invention for classifying an individual as having bladder cancer or recurrent bladder cancer or being at risk of developing bladder cancer, for typing a urine sample from an individual or for determining for determining a treatment strategy for an individual. Preferably said classifying an individual, typing a urine sample or determining a treatment strategy is performed with a method according to the invention as disclosed herein.

Classifying an individual as having bladder cancer or recurrent bladder cancer or being at risk of developing bladder cancer according to the invention preferably comprises a further diagnostic test for the presence of bladder cancer or recurrent bladder cancer or for being at risk of developing bladder cancer. One such further test preferably comprises cystoscopy with or without subsequent analyzing a biopsy from the bladder of the individual. The further diagnosis can confirm the presence of bladder cancer or not.

Urine can be used to screen a population for the presence of individuals with a disease. The non-invasive collection is one of the attractive features. Urine diagnostics have been suggested for various diseases including cancer. Urine is for instance used to detect bladder cancer. Large scale screening or screening of patients at risk of bladder cancer (e.g. patients with hematuria) is typically a first step. Individuals that tested “positive” are typically further tested with one or more further confirmatory diagnostic methods. In one embodiment the invention thus provides a method for screening a population for the presence of individuals that have bladder cancer or recurrent bladder cancer or being at risk of developing bladder cancer, the method comprising determining DNA methylation of at least a first gene or a promoter region thereof and a second gene or a promoter region thereof in a urine samples from individuals in said population, wherein said genes are selected from the group consisting of GHSR, MAL, FAM19A4, PHACTR3, PRDM14, SST, ZIC1, miR-129, miR-148 and miR-935 and classifying said individuals of said population as having bladder cancer or recurrent bladder cancer or being at risk of developing bladder cancer based on said DNA methylation. An individual that tested positive with such a method is preferably further tested with a further diagnostic test for bladder cancer, preferably by cystoscopy with or without subsequent analyzing a bladder biopsy.

The non-invasive character of the analysis also makes a method of the invention suited to follow an individual that is being treated or has been treated for bladder cancer to determine the effect of the treatment or a determine a risk of recurrence of tumor in a non-invasive way. Patients that test positive in a follow-up setting after treatment of bladder cancer are tested with a further diagnostic test for bladder cancer, preferably by cystoscopy with or without subsequent analyzing of a bladder biopsy.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The term “individual” refers to any animal, such as a mammal, including, but not limited to humans, non-human primates, canines, felines, rodents, etc. In a preferred embodiment an individual is a human individual.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: Box plots showing the methylation levels of all fourteen markers (CADM1, FAM19A4, GHSR, MAL, PHACTR3, PRDM14, SST, ZIC1, miR-124-2, miR-129, miR-137, miR-148, miR-181 and miR-935) represented by the log 2-transformed Ct ratios (y-axis) in controls (blue) and bladder cancer patients (orange). P-values were calculated using the Mann-Whitney U test

FIG. 2: Receiver operator characteristic (ROC) curves using the cycle threshold (CT) ratio values of GHSR (a), MAL (b), PRDM14 (c) and SST (d) which proved to have the highest areas under the curve (AUC) for the diagnosis of bladder cancer.

FIG. 3: Receiver operator characteristic (ROC) curves using the cycle threshold (CT) ratio values of all markers except for the four with the highest areas under the curve (GHSR, MAL, PRDM, and SST) for the diagnosis of bladder cancer.

FIG. 4: Box plots showing the methylation levels of GHSR and MAL represented by the log 2-transformed Ct ratios (y-axis) in normal bladder tissue (n=10, blue) and bladder cancer tissue (n=21, orange). EDTA=Ethylenediaminetetraacetic acid; RT=room temperature Penstrep=Penicillin Streptomycin

FIG. 5: Schematic overview of sample handling in volunteers (n=3) and patients (non-small cell lung cancer n=10, bladder cancer, n=10). EDTA=Ethylenediaminetetraacetic acid; RT=room temperature Penstrep=Penicillin Streptomycin.

FIG. 6: DNA yield of bisulfite modified DNA measured by mean log fold change of 6-actin (ACTB) compared to t=0 after storing urine at room temperature, 4° C., −20° C. and −80° C. with and without preserving agents. Error bars represent the standard deviation between 3 urine samples derived from different donors. RT=room temperature; ACTB=6-actin; Penstrep=Penicillin Streptomycin; EDTA=Ethylenediaminetetraacetic acid.

FIG. 7: DNA integrity, derived from urine samples stored at room temperature and 4° C., after 7 days with and without preserving agents. DNA integrity was measured by the log fold change of β-actin (ACTB) expression at various conditions compared to day 0. Results of the post-hoc analysis of DNA integrity at room temperature can be found in Table 12. EDTA=Ethylenediaminetetraacetic acid; PS=Penicillin Streptomycin.

FIG. 8: Correlation plots showing the correlation between ΔCT of β-actin (ACTB) and the ΔCT of methylated RASSF1A with or without various preserving agents (additives) at room temperature and at 4° C. The outliers are marked with A-E. EDTA=Ethylenediaminetetraacetic acid; ACTB=β-actin; Pen strep=Penicillin Streptomycin.

FIG. 9: A-J. Promoter sequences, CpG rich sequences and (part of) exon 1 of GHSR, MAL, FAM19A4, PHACTR3, PRDM14, SST, ZIC1, miR-129, miR-148 and miR-935. Exon 1 is in upper case. The promoter sequence containing CpG rich sequences is in lower case. CpG rich regions, or CpG islands, are shaded in grey.

### EXAMPLES

### Example 1 Methylation Signature for the Diagnosis of Bladder Cancer in Urine

Materials and Methods

Patients and Urine Samples

A total of 147 urine samples were used for this study, of which 72 urine samples were from BC patients, and the remaining 75 urine samples were collected from patients with no history of a urinary tract malignancy as controls. The controls were age and gender matched. Two patients and two controls were excluded based on insufficient DNA quality for qMSP analysis (see section qMSP). Patient characteristics of 73 BC patients with valid qMSP results are shown in Table 3. Of the BC samples, most patients had NMIBC (63%), most were grade 3 tumours (53%) and the majority consisted of primary tumours (64%). Samples of BC patients were collected at the department of Urology in the VU University Medical Center (n=49) between October 2014 and October 2017. Urine samples were collected prior to cystoscopy or prior to transurethral resection of a bladder tumour (TURBT) and stored at −80° C. within 4 hours after collection.

Informed written consent was obtained from all participants and the research protocol was approved by the medical ethical committee of the VU University Medical Center (2016.425). Additionally, urine specimens from 75 controls and 23 BC patients (retrieved from a biobank) and tissue specimens of 10 non-malignant bladder and 21 bladder cancers (retrieved from the Pathology archive) were used according to The Code for Proper Secondary Use of Human Tissue in the Netherlands (http://www.federa.org/).

DNA isolation and bisulphite conversion Genomic DNA was extracted from 2-40 mL full urine (depending on the original volume) using the Quick-DNA™ Urine Kit (Zymo Research, Orange, Calif., U.S.A.)

according to the manufacturer's protocol. Isolated DNA was converted with bisulphite using the EZ DNA Methylation™ kit (Zymo Research, Orange, Calif., U.S.A.).

Quantitative Methylation Specific PCR (qMSP)

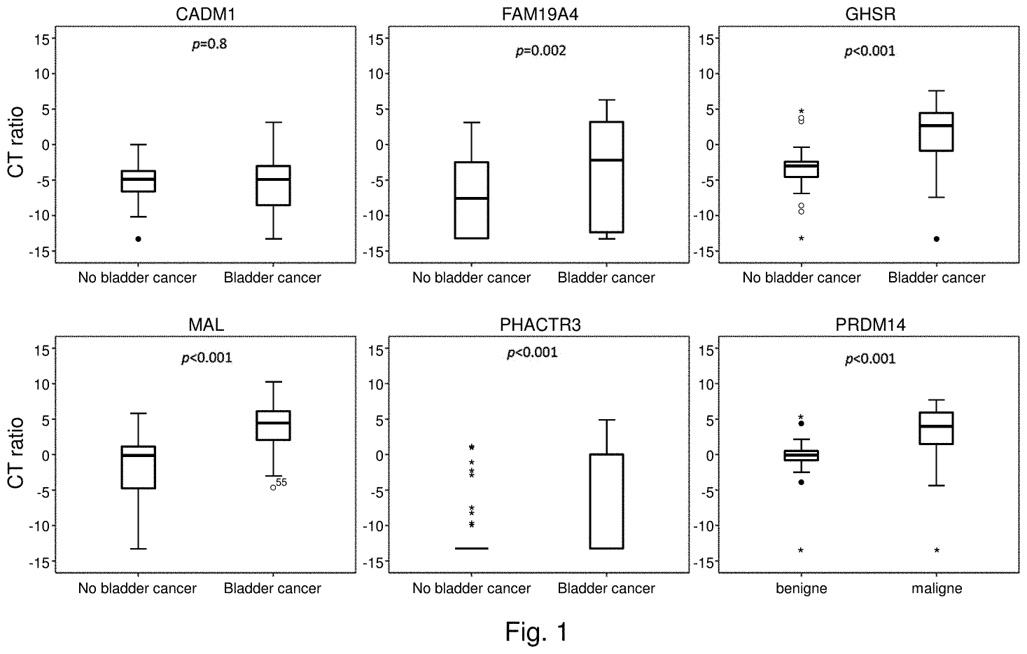

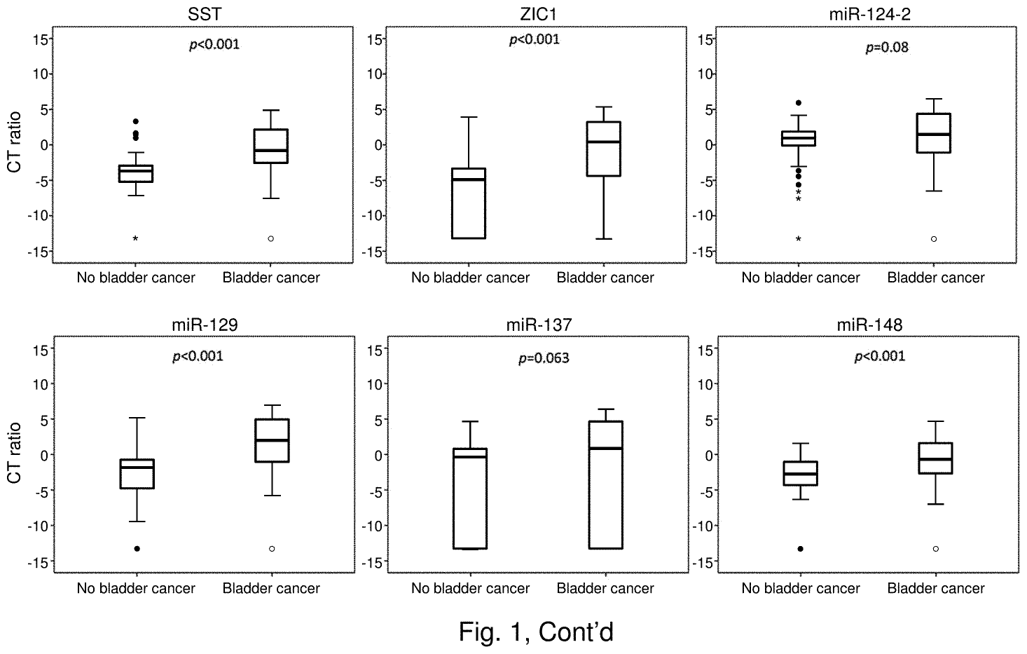

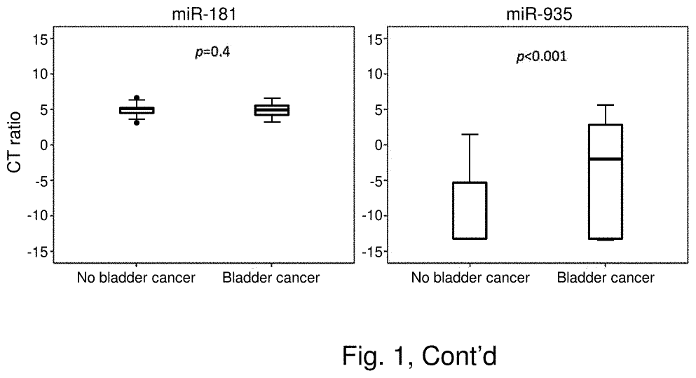

Bisulphite converted DNA was then used as template for DNA methylation analysis. Multiplex qMSP reactions were performed for a total of 14 genes (FIG. 1). Primers were designed to specifically amplify methylated bisulphite-treated DNA and combined in multiplex assays targeting 2-3 genes with ACTB as a sample quality control, as previously described (Wilting 2016; Steenbergen 2013; Verlaat 2017; Hubers 2015). Primer and probe sequences are shown in table 2. Multiplex qMSP was performed using 50 ng of bisulphite-converted DNA and 200-300 nmol/L of each primer and fluorescent dye-labeled probe, on the ABI 7500 Fast Real-Time PCR System (Applied Biosystems, USA).

Modified DNAs of the BC cell lines RT-112, TCC-SUP and J82 were used as positive controls and H2O as negative control. Samples with an ACTB CT>32, were considered unsuitable for DNA methylation analysis, which resulted in the exclusion of two patients and two controls. Methylation values of the targets were normalized to the reference gene ACTB, using the comparative CT method (2^{-ΔCT}×100) (Schmittgen 2008).

Statistical Analysis

Categorical data was summarized with frequency and percentage, and continuous data with mean, median, first and third quartiles. The χ²-test was used to compare categorical data between groups, and the independent samples t-test was used to compare means of continuous data between groups. CT-ratios of BC patients were compared to those of controls using the Mann-Whitney U test. All CT-ratios were ² log transformed.

A receiver operating characteristic (ROC) curve was plotted with the ratio value for each marker and the area under the curve (AUC) was determined. Cut-offs were obtained by Youden's J index (Schisterman 2008; Youden 1950). To find the best combination of methylation markers multivariate logistic regression was used with two selection procedures: forward and backward selection. For the forward procedure the p-value for entry was ≤0.05. Additionally, the seven markers with the highest AUC were tested with a backward selection procedure (p-value for removal>0.05). The combination of markers that were identified in this manner, were thereafter considered as a single test. The final combination of these markers was considered positive if at least one of the individual markers was positive (believe-the-positive) (Marshall 1989). The difference in sensitivities between several BC subgroups was analyzed with the χ²-test.

To estimate the accuracy of the diagnostic value of the markers individually as well of from the final model (combination of markers), leave-one-out cross validation (LOOCV) was performed. LOOCV was performed with R statistical software (version 3.4.2). Remaining statistical analysis was performed with SPSS software (SPSS 22.0, IBM Corp., Armonk, N.Y., U.S.A.). Tests were two-sided and the significance level was set at 0.05 for all tests.

Results

DNA Hypermethylation Analysis: Discriminative Capability of Individual Genes

The methylation levels of the markers FAM19A4, GHSR, MAL, PHACTR3, PRDM14, SST, ZIC1, miR-129, miR-148 and miR-935 were significantly higher in BC patients compared to controls. The methylation levels of the markers CADM1, miR-137 and miR-181, did not differ significantly between BC patients and controls (FIG. 1).

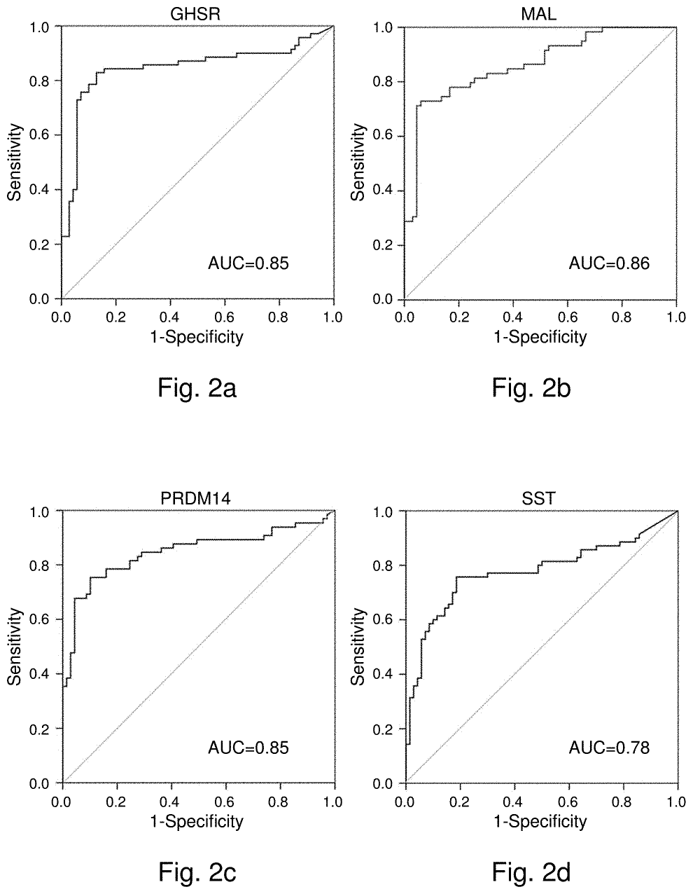

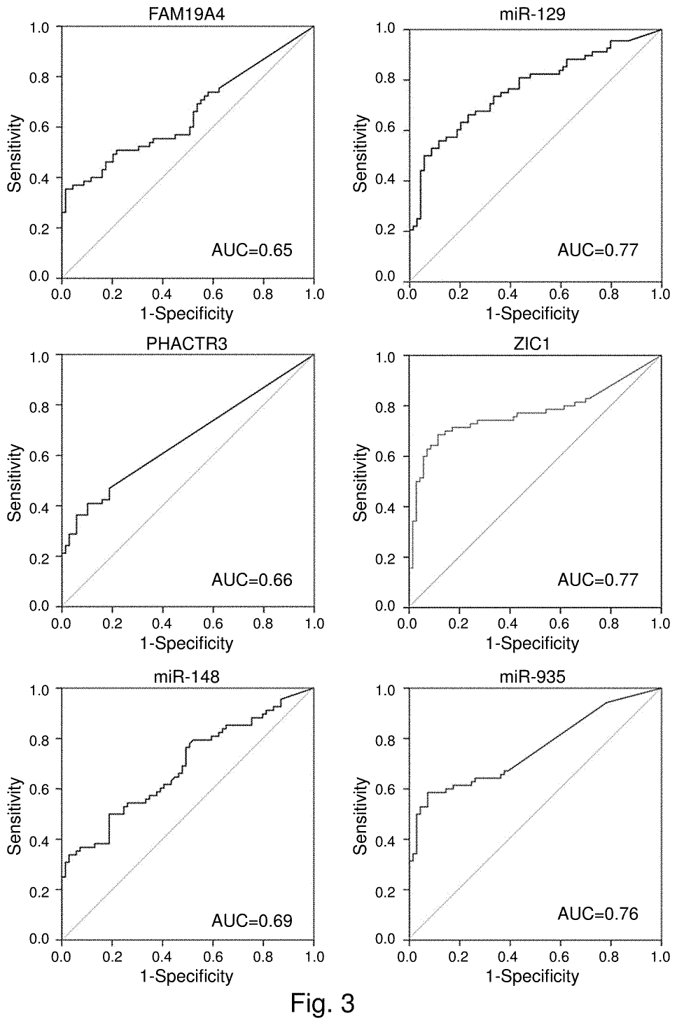

The AUC, sensitivity and specificity for each marker are presented in Table 4. The ROC-curves of the four markers with the highest AUC are shown in FIG. 2. Highest sensitivities were obtained for GHSR (83%), SST (76%), PRDM14 (75%) and MAL (73%), at corresponding specificities of 87%, 81%, 90% and 94%, respectively. The ROC curves of the remaining markers are shown in FIG. 3. The performances of each marker per individual are presented in Tables 5-6. LOOCV yielded similar diagnostic accuracy (percentage of correct diagnosis) for almost all individual markers, except for miR-148 (Table 7). For miR-148 the diagnostic accuracy was lower in the LOOCV (58% LOOCV versus 66% original analysis).

DNA Hypermethylation Analysis: Discriminative Capability of Multiple Genes

To determine which markers are complementary to each other, and thereby define the optimal gene panel, a forward and backward selection procedure was used. Only the markers that were significantly higher in BC patients were tested. From both the forward and the backward selection procedure the combination of GHSR and MAL was identified as the optimal panel. Positive DNA methylation of at least one of these two markers (‘believe-the-positive’) resulted in a sensitivity of 92% (95% confidence interval (CI): 86-99) and specificity of 85% (95% CI: 76-94). LOOCV of the combination GHSR and MAL yielded similar diagnostic accuracy as the original analysis (diagnostic accuracy of 89% in the original analysis versus 89% in LOOCV).

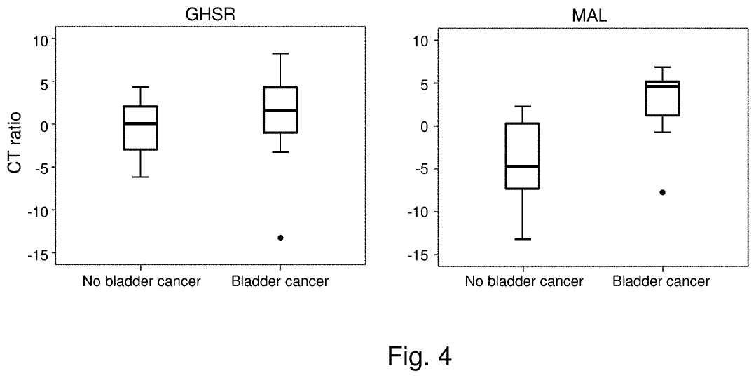

To verify that GHSR and MAL hypermethylation originates from the tumor, tissue specimens were also analysed. Both GHSR and MAL showed higher methylation levels in BC tissues compared to benign bladder tissues (FIG. 4).

The sensitivity and specificity of all combinations of marker for positive DNA methylation of at least one of the two markers are shown in Tables 9 and 10.

Methylation Levels in Relation to Tumour T-Stage, Recurrence (Yes/No), Tumour Grade and Gender

For the 10 methylation markers that were significantly higher in BC patients compared to controls, sensitivities in relation to tumour stage are presented in table 8. All markers, except MAL, had a higher sensitivity for ≥T2 tumours (n=25) as compared to Ta-T1 tumours (n=42). The combination of GHSR and MAL performed better in Ta-T1 tumours, although the difference was not statistically significant (p=0.6). Surprisingly, sensitivity for all tumour grades and for both primary tumors and recurrent cancer are higher than could be achieved with any individual marker. Sensitivities of single markers were mostly higher for grade 3 tumours (n=37), as compared to grade 1-2 tumours (n=29). Also the combination of GHSR and MAL showed a higher sensitivity in grade 3 tumours, although the difference was not statistically significant (p=0.8).

The sensitivity for the detection of primary versus recurrent tumours varied per marker, but did not differ for the marker combination GHSR and MAL. However, the GHSR/MAL combination displayed significantly higher sensitivity in male patients (n=52) compared to female (n=18) patients (98% versus 73%, p=0.001), but no difference in specificity (82% versus 93%, p=0.3). A similar trend was seen upon subdivision per tumor grade.

TABLE 3
Baseline and tumour characteristics of patients with bladder cancer
(n = 70) and controls (n = 73).

| | Bladder | Non-malignant | |
|---|---|---|---|
| | cancer (n = 70) | (n = 73) | |
| Characteristic | n, % | n, % | p-value |
| Sex | | | |
| Male | 52 (74) | 58 (80) | 0.5 |
| Female | 18 (26) | 15 (21) | |
| Age, yr | | | |
| Median (IQR) | 73 (67-77) | 70 (65-75) | 0.4 |
| Primary/recurrence | | | |
| primary | 43 (64) | — | — |
| recurrent | 24 (36) | | |
| missing n = 3 | | | |
| Stage | | | |
| Ta | 33 (48) | — | — |
| T1 | 9 (13) | | |
| ≥T2 | 25 (36) | | |
| Tis | 2 (2.8) | | |
| Tx | 1 (1.4) | | |
| Grade | | | |
| 1 | 8 (11) | — | — |
| 2 | 21 (30) | | |
| 3 | 37 (53) | | |
| Gx | 2 (2.9) | | |
| CIS only | 2 (2.9) | | |
| Concomitant CIS | | | |
| No | 69 (99) | — | — |
| Yes | 1 (1.4) | | |

IQR = interquartile range;
Gx = grade not specified;
CIS = carcinoma in situ

TABLE 4
DNA hypermethylation markers evaluated as a binary marker, ordered by
decreasing area under the curve (AUC). Cut-offs were determined
with Youden's J index (Youden 1950).

| Marker | AUC | 95% CI | Sens | 95% CI | Spec | 95% CI |
|---|---|---|---|---|---|---|
| MAL | 0.86 | 0.80-0.93 | 0.73 | 0.62-0.84 | 0.94 | 0.88-1.00 |
| GHSR | 0.85 | 0.77-0.92 | 0.83 | 0.74-0.92 | 0.87 | 0.76-0.94 |
| PRDM14 | 0.85 | 0.78-0.92 | 0.75 | 0.65-0.86 | 0.90 | 0.83-0.97 |
| SST | 0.78 | 0.70-0.86 | 0.76 | 0.66-0.86 | 0.81 | 0.72-0.91 |
| ZIC1 | 0.77 | 0.69-0.86 | 0.69 | 0.58-0.79 | 0.89 | 0.81-0.96 |
| miR-129 | 0.77 | 0.69-0.85 | 0.50 | 0.38-0.62 | 0.94 | 0.89-1.00 |
| miR-935 | 0.76 | 0.68-0.84 | 0.59 | 0.47-0.70 | 0.93 | 0.87-0.99 |
| miR-148 | 0.69 | 0.60-0.78 | 0.50 | 0.38-0.62 | 0.81 | 0.72-0.90 |
| PHACTR3 | 0.66 | 0.57-0.76 | 0.41 | 0.29-0.53 | 0.90 | 0.83-0.97 |
| FAM19A4 | 0.65 | 0.56-0.75 | 0.35 | 0.24-0.47 | 0.99 | 0.96-1.00 |

AUC = area under the curve; CI = confidence interval; Sens = sensitivity; Spec = specificity

TABLE 5
Diagnostic accuracy of all individual markers per patient sample.

| | | | | | Age | | | | miR- | miR- | | | | | |
|---|---|---|---|---|---|---|---|---|---|---|---|---|---|---|---|
| No. | Sex | T-cat | G | P/R | (yr) | GHSR | SST | ZIC1 | 129 | 148 | miR935 | FAM19α4 | PHACTR3 | PRDM14 | MAL |
| 1 | m | pTa | 3 | R | 61 | | | | | | | | | | — |
| 2 | m | pTa | 2 | P | 79 | | | | | | | | | | — |
| 3 | m | pT1 | 3 | P | 67 | | | | | | | | | | — |
| 4 | f | pTa | 3 | R | 69 | | | | | | | | | | — |
| 5 | m | pTa | 2 | P | 64 | | | | | | | | | | — |
| 6 | m | pTa | 1 | R | 71 | | | | | | | — | — | — | — |
| 7 | f | pTa | 1 | P | 78 | | | | | | | | | | |
| 8 | m | pTa | 1 | R | 68 | | | | | | | | | | |
| 9 | f | pTa | 1 | R | 86 | | | | | | | | | | |
| 10 | m | pTa | 1 | R | 76 | | | | | | | — | — | — | |
| 11 | m | pTa | 2 | R | 64 | | | | | | | | | | |
| 12 | m | pTa | 2 | P | 73 | | | | | | | | | | |
| 13 | m | pT1 | 3 | P | 59 | | | | | | | | | | |
| 14 | m | pTa | 3 | P | 66 | | | | | | | | | | |
| 15 | m | pT1 | 3 | P | 75 | | | | | | | | | | |
| 16 | m | pT1 | 3 | P | 71 | | | | | | | | | | |
| 17 | m | pTa | 2 | P | 82 | | | | | | | | | | |
| 18 | m | pT1 | 1 | P | 74 | | | | | | | | | | |
| 19 | m | pT1 | 3 | P | 84 | | | | | | | | | | |
| 20 | m | pTa | 3 | P | 80 | | | | | | | | | | |
| 21 | f | pTa | 2 | P | 83 | | | | | | | | | | |
| 22 | m | pTa | 2 | P | 80 | | | | | | | | | | |
| 23 | m | pTa | 1 | R | 69 | | | | | | | | | | |
| 24 | m | pTa | 1 | R | 60 | | | | | | | | | | |
| 25 | m | pTa | 2 | P | 67 | | | | | | | | | | |
| 26 | m | pTa | 3 | R | 79 | | | | | | | | | | |
| 27 | m | pTa | 3 | P | 60 | | | | | | | | | | |
| 28 | m | pTa | 2 | R | 74 | | | | | | | | | | |
| 29 | m | pTa | 2 | R | 73 | | | | | | | | | | |
| 30 | f | pTa | 2 | R | 70 | | | | | | | | | | |
| 31 | m | pTa | 2 | P | 91 | | | | | | | | | | |
| 32 | m | pTa | 3 | R | 54 | | | | | | | | | | |
| 33 | m | pT1 | 2 | P | 59 | | | | | | | | | | |
| 34 | f | pT2 | 2 | P | 59 | | | | | | | | | | |
| 35 | m | pT4 | 3 | P | 76 | | | | — | — | | — | — | — | — |
| 36 | m | pT2 | 3 | P | 68 | | | | | | | | | | |
| 37 | m | pT4 | 3 | R | 67 | | | | | | | | | | |
| 38 | m | pT2 | 3 | P | 76 | | | | | | | | | | |
| 39 | m | pT2 | Gx | R | 91 | | | | | | | | | | |
| 40 | m | pT2 | 3 | | 55 | | | | | | | | | | |
| 41 | m | pT2 | 3 | P | 67 | | | | | | | | | | |
| 42 | m | pT2 | 3 | P | 68 | | | | | | | | | | |
| 43 | m | pT2 | 3 | R | 69 | | | | | | | | | | |
| 44 | m | pT2 | 3 | | 72 | | | | | | | | | | |
| 45 | m | pT2 | 3 | R | 73 | | | | | | | | | | |
| 46 | m | pT2 | 3 | P | 65 | | | | | | | | | | |
| 47 | m | pT3 | 3 | P | 75 | | | | | | | | | | |
| 48 | f | pT2 | 3 | P | 90 | | | | | | | | | | |
| 49 | f | pT2 | 3 | | 92 | | | | | | | | | | |
| 50 | m | pT2 | 3 | P | 69 | | | | | | | | | | |
| 51 | m | pT2 | 3 | P | 46 | | | | | | | | | | |
| 52 | m | pT2 | 3 | P | 79 | | | | | | | | | | |
| 53 | m | pT2 | 3 | P | 74 | | | | | | | | | | |
| 54 | m | pT2 | 3 | P | 78 | | | | | | | | | | |
| 55 | f | pT3 | 3 | P | 67 | | | | | | | | | | |
| 56 | f | pTis | 3 | R | 78 | | | | | | | | | | |
| 57 | m | pTis | CIS | R | 63 | | | | | | | | | | |
| 58 | m | — | CIS | R | 73 | | | | | | | | | | |
| 59 | f | pTa | | R | 70 | | | | | | | | | | |
| 60 | m | pTa | 3 | P | 53 | | | | | | | | | | |
| 61 | f | pTa | 2 | P | 75 | | | | | | | | | | — |
| 62 | f | pTa | 2 | P | 45 | | | | | | | — | — | — | — |
| 63 | f | pTa | 2 | P | 75 | | | | | | | | | | |
| 64 | f | pT1 | 2 | P | 79 | | | | | | | | | | |
| 65 | m | pTa | 2 | P | 73 | | | | | | | | | | |
| 66 | m | pTa | 2 | R | 49 | | | | | | | | | | |
| 67 | f | pT2 | 2 | P | 73 | | | | | | | | | | |
| 68 | f | pT2 | 3 | P | 72 | | | | — | — | | — | | — | — |
| 69 | m | pT2 | 3 | P | 77 | | | | | | | | | | |
| 70 | f | pTa | 3 | R | 74 | | | | | | | | | | |
| 71 | m | pTa | 3 | P | 50 | — | — | — | — | — | — | — | — | — | — |
| 72 | m | pTa | 2 | P | 54 | — | — | — | — | — | — | — | — | — | — |

F = female; M = male; Grey: Hypermethylation in urine; White: No Hypermethylation; —: No data due to high ACTB value.

TABLE 6
Diagnostic accuracy of all individual markers per control sample.

| number | Sex | Age (yr) | GHSR | SST | ZIC1 | miR-129 | miR-148 | miR-935 | FAM19A4 | PHACTR3 | PRDM14 | MAL |
|---|---|---|---|---|---|---|---|---|---|---|---|---|
| 1 | M | 63 | | | | — | — | — | — | — | — | — |
| 2 | F | 71 | | | | | | | | | | |
| 3 | M | 71 | | | | | | | | | | |
| 4 | M | 60 | | | | | | | | | | |
| 5 | M | 62 | | | | | | | | | | |
| 6 | M | 73 | | | | | | | | | | |
| 7 | M | 68 | | | | | | | | | | |
| 8 | M | 75 | | | | | | | | | | |
| 9 | M | 72 | | | | | | | | | | |
| 10 | M | 69 | | | | | | | | | | — |
| 11 | M | 76 | | | | | | | | | | — |
| 12 | M | 68 | | | | | | | — | — | — | — |
| 13 | M | 51 | | | | | | | — | — | — | — |
| 14 | M | 80 | | | | | | | | | | |
| 15 | F | 71 | | | | | | | | | | |
| 16 | M | 68 | | | | | | | | | | |
| 17 | F | 52 | | | | | | | | | | |
| 18 | M | 72 | | | | | | | | | | |
| 19 | F | 80 | | | | | | | | | | |
| 20 | M | 65 | | | | | | | | | | |
| 21 | M | 60 | | | | | | | | | | |
| 22 | F | 73 | | | | | | | | | | |
| 23 | M | 54 | | | | | | | | | | |
| 24 | M | 72 | | | | | | | | | | |
| 25 | F | 81 | | | | | | | | | | |
| 26 | M | 78 | | | | | | | | | | |
| 27 | M | 72 | | | | | | | | | | |
| 28 | M | 81 | | | | | | | | | | |
| 29 | M | 63 | | | | | | | | | | |
| 30 | F | 63 | | | | | | | | | | |
| 31 | M | 73 | | | | | | | | | | |
| 32 | M | 72 | | | | | | | | | | |
| 33 | F | 80 | | | | | | | | | | |
| 34 | M | 72 | | | | | | | | | | |
| 35 | M | 55 | | | | | | | | | | |
| 36 | M | 65 | | | | | | | | | | |
| 37 | M | 75 | | | | | | | | | | |
| 38 | M | 70 | | | | | | | | | | |
| 39 | M | 80 | | | | | | | | | | |
| 40 | M | 71 | | | | | | | | | | |
| 41 | F | 76 | | | | | | | | | | |
| 42 | F | 55 | | | | | | | | | | |
| 43 | M | 72 | | | | | | | | | | |
| 44 | M | 60 | | | | | | | | | | |
| 45 | M | 75 | | | | | | | | | | |
| 46 | F | 83 | | | | | | | | | | |
| 47 | M | 70 | | | | | | | | | | |
| 48 | M | 68 | | | | | | | | | | |
| 49 | M | 73 | | | | | | | | | | |
| 50 | F | 84 | | | | | | | | | | |
| 51 | M | 69 | | | | | | | | | | |
| 52 | M | 82 | | | | | | | | | | |
| 53 | F | 63 | | | | | | | | | | |
| 54 | M | 85 | | | | | | | | | | |
| 55 | M | 76 | | | | | | | | | | |
| 56 | M | 84 | | | | | | | | | | |
| 57 | M | 54 | | | | | | | | | | |
| 58 | M | 48 | | | | | | | | | | |
| 59 | M | 70 | | | | | | | | | | |
| 60 | F | 67 | | | | | | | | | | |
| 61 | M | 85 | | | | | | | | | | |
| 62 | M | 61 | | | | | | | | | | |
| 63 | F | 82 | | | | | | | | | | |
| 64 | M | 63 | | | | | | | | | | |
| 65 | M | 69 | | | | | | | | | | |
| 66 | M | 67 | | | | | | | | | | |
| 67 | M | 70 | | | | | | | | | | |
| 68 | M | 68 | | | | | | | | | | |
| 69 | M | 65 | | | | | | | | | | |
| 70 | M | 69 | | | | | | | | | | |
| 71 | M | 71 | — | — | — | — | — | — | — | | | — |
| 72 | M | 67 | — | — | — | — | — | — | — | | | — |
| 73 | F | 77 | — | — | — | — | — | — | — | — | — | — |
| 74 | M | 70 | — | — | — | — | — | — | — | — | — | — |
| 75 | M | 67 | — | — | — | — | — | — | — | — | — | |

F = female; M = male; Grey: Hypermethylation in urine; White: No Hypermethylation; —: No data due to high ACTB value.

TABLE 7
Results of leave one out cross validation (LOOCV) showing accuracy
(percentage of correct diagnosis) per marker and for the finally selected
combination of GHSR and MAL.

| | Original | | | LOOCV | | |
|---|---|---|---|---|---|---|
| Marker | Accuracy | Sens | Spec | Accuracy | Sens | Spec |
| FAM19A4 | 0.68 | 0.35 | 0.99 | 0.66 | 0.34 | 0.96 |
| GHSR | 0.85 | 0.83 | 0.87 | 0.85 | 0.83 | 0.87 |
| MAL | 0.84 | 0.73 | 0.94 | 0.83 | 0.71 | 0.94 |
| miR-129 | 0.72 | 0.50 | 0.94 | 0.69 | 0.50 | 0.88 |
| miR-148 | 0.66 | 0.50 | 0.81 | 0.58 | 0.34 | 0.81 |
| miR-935 | 0.76 | 0.59 | 0.93 | 0.74 | 0.57 | 0.91 |
| PHACTR3 | 0.66 | 0.41 | 0.90 | 0.64 | 0.36 | 0.90 |
| PRDM14 | 0.83 | 0.75 | 0.90 | 0.83 | 0.75 | 0.90 |
| SST | 0.79 | 0.76 | 0.81 | 0.79 | 0.76 | 0.81 |
| ZIC1 | 0.79 | 0.69 | 0.89 | 0.78 | 0.67 | 0.89 |
| GHSR + MAL | 0.89 | 0.92 | 0.86 | 0.89 | 0.92 | 0.86 |

LOOCV = leave one out cross validation; Sens = sensitivity; Spec = specificity

TABLE 8
Sensitivities of methylation of the genes that were significantly associated with the presence of
bladder cancer, as well as the sensitivities of the finally chosen methylation assay (GHSR + MAL).
Sensitivity of methylation, % (95% CI)

| | | | | | | | | | | | GHSR + |
|---|---|---|---|---|---|---|---|---|---|---|---|
| Subgroups | GHSR | SST | ZIC1 | miR-129 | miR-148 | miR-935 | FAM19A4 | PHACTR3 | PRDM14 | MAL | MAL |
| All | 83 | 76 | 69 | 50 | 50 | 59 | 35 | 41 | 75 | 73 | 92 |
| | (74-92) | (66-86) | (58-79) | (38-62) | (38-62) | (47-70) | (24-47) | (29-53) | (65-86) | (62-84) | (86-99) |
| T-category | | | | | | | | | | | |
| Ta/T1 | 81 | 71 | 64 | 45 | 50 | 57 | 31 | 33 | 74 | 76 | 95 |
| | (69-93) | (58-85) | (50-79) | (30-60) | (35-65) | (42-72) | (16-45) | (19-48) | (61-88) | (61-90) | (88-100) |
| ≥T2 | 88 | 84 | 76 | 61 | 57 | 60 | 44 | 54 | 78 | 70 | 92 |
| | (75-100) | (70-98) | (59-93) | (41-81) | (36-77) | (41-79) | (23-64) | (34-74) | (61-95) | (51-88) | (81-100) |
| Prim/rec | | | | | | | | | | | |
| Primary | 79 | 70 | 70 | 56 | 51 | 54 | 43 | 42 | 75 | 80 | 92 |
| | (67-91) | (56-84) | (56-84) | (41-71) | (26-67) | (39-68) | (27-58) | (26-57) | (62-88) | (67-93) | (84-100) |
| Recurrence | 88 | 83 | 63 | 38 | 46 | 63 | 18 | 41 | 73 | 62 | 92 |
| | (74-100) | (68-98) | (43-82) | (18-57) | (26-66) | (43-82) | (2-34) | (20-62) | (54-91) | (41-83) | (81-100) |
| Grade | | | | | | | | | | | |
| 1-2 | 92 | 66 | 55 | 38 | 52 | 48 | 19 | 19 | 65 | 70 | 93 |
| | (81-100) | (48-83) | (37-73) | (20-56) | (34-70) | (30-67) | (4-34) | (4-34) | (47-84) | (53-88) | (83-100) |
| 3 | 89 | 84 | 78 | 63 | 54 | 65 | 49 | 56 | 86 | 81 | 94 |
| | (79-99) | (72-96) | (65-92) | (47-79) | (38-71) | (50-80) | (32-65) | (39-72) | (74-97) | (67-95) | (87-100) |
| Sex | | | | | | | | | | | |
| Male | 92 | 87 | 81 | 57 | 61 | 71 | 58 | 49 | 82 | 78 | 98 |
| | (85-100) | (77-96) | (70-92) | (43-71) | (47-74) | (59-84) | (44-72) | (35-63) | (71-93) | (66-90) | (94-100) |
| Female | 56 | 44 | 33 | 29 | 17 | 22 | 19 | 18 | 56 | 57 | 73 |
| | (33-79) | (22-67) | (12-55) | (8-51) | (0-36) | (3-41) | (0-38) | (0-36) | (32-81) | (31-83) | (51-96) |

CI = confidence interval; Prim = primary; rec = recurrence

TABLE 9
Sensitivity of all combinations of markers for
positive DNA methylation of at least one of the two markers.

| | ZIC1 | SST | GHSR | miR129 | miR148 | miR935 | FAM19A4 | PHACTR3 | PRDM14 | MAL |
|---|---|---|---|---|---|---|---|---|---|---|
| ZIC1 | 68.6% | 80.0% | 84.3% | 73.5% | 76.5% | 77.1% | 75.4% | 75.8% | 81.5% | 81.4% |
| SST | 80.0% | 75.7% | 87.1% | 82.4% | 77.9% | 81.4% | 81.5% | 81.8% | 86.2% | 91.5% |
| GHSR | 84.3% | 87.1% | 82.9% | 86.8% | 86.8% | 87.1% | 86.2% | 86.4% | 86.2% | 91.5% |
| miR129 | 73.5% | 82.4% | 86.8% | 50.0% | 69.1% | 67.6% | 55.4% | 56.9% | 76.9% | 72.9% |
| miR148 | 76.5% | 77.9% | 86.8% | 69.1% | 50.0% | 69.1% | 61.5% | 64.6% | 80.0% | 79.7% |
| miR935 | 77.1% | 81.4% | 87.1% | 67.6% | 69.1% | 58.6% | 61.5% | 63.6% | 81.5% | 83.1% |
| FAM19A4 | 75.4% | 81.5% | 86.2% | 55.4% | 61.5% | 61.5% | 35.4% | 49.2% | 75.4% | 75.9% |
| PHACTR3 | 75.8% | 81.8% | 86.4% | 56.9% | 64.6% | 63.6% | 49.2% | 40.9% | 78.5% | 77.6% |
| PRDM14 | 81.5% | 86.2% | 86.2% | 76.9% | 80.0% | 81.5% | 75.4% | 78.5% | 75.4% | 82.8% |
| MAL | 81.4% | 91.5% | 91.5% | 72.9% | 79.7% | 83.1% | 75.9% | 77.6% | 82.8% | 72.9% |

TABLE 10
Specificity of all combinations of markers for
positive DNA methylation of at least one of the two markers.

| | ZIC1 | SST | GHSR | miR129 | miR148 | miR935 | FAM19A4 | PHACTR3 | PRDM14 | MAL |
|---|---|---|---|---|---|---|---|---|---|---|
| ZIC1 | 88.6% | 77.1% | 84.3% | 89.9% | 73.9% | 85.5% | 89.6% | 83.6% | 88.1% | 87.7% |
| SST | 77.1% | 81.4% | 78.6% | 81.2% | 65.2% | 79.7% | 80.6% | 74.6% | 77.6% | 78.5% |
| GHSR | 84.3% | 78.6% | 87.1% | 88.4% | 71.0% | 87.0% | 88.1% | 82.1% | 85.1% | 86.2% |
| miR129 | 89.9% | 81.2% | 88.4% | 94.2% | 75.4% | 89.9% | 94.0% | 86.6% | 89.6% | 92.3% |
| miR148 | 73.9% | 65.2% | 71.0% | 75.4% | 81.2% | 75.4% | 79.1% | 73.1% | 71.6% | 73.8% |
| miR935 | 85.5% | 79.7% | 87.0% | 89.9% | 75.4% | 92.8% | 92.5% | 83.6% | 85.1% | 87.7% |
| FAM19A4 | 89.6% | 80.6% | 88.1% | 94.0% | 79.1% | 92.5% | 98.6% | 88.4% | 89.9% | 92.3% |
| PHACTR3 | 83.6% | 74.6% | 82.1% | 86.6% | 73.1% | 83.6% | 88.4% | 89.9% | 82.6% | 86.2% |
| PRDM14 | 88.1% | 77.6% | 85.1% | 89.6% | 71.6% | 85.1% | 89.9% | 82.6% | 89.9% | 87.7% |
| MAL | 87.7% | 78.5% | 86.2% | 92.3% | 73.8% | 87.7% | 92.3% | 86.2% | 87.7% | 93.9% |

### Example 2. Protocol for Urine Collection and Storage Prior to DNA Methylation Analysis

Materials and Methods

This pilot and patients sample study was approved by the Medical Ethics Review Committee of the VU University Medical Centre and informed written consent was obtained from all participants.

Sample Collection and Storage

Pilot Study

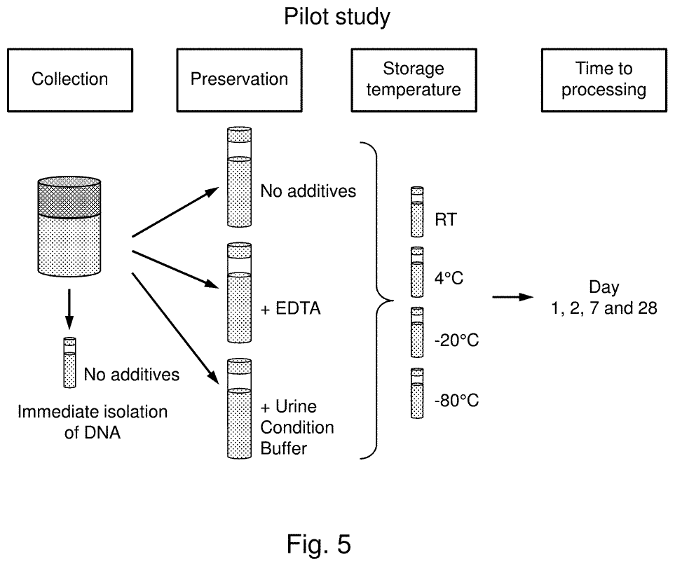

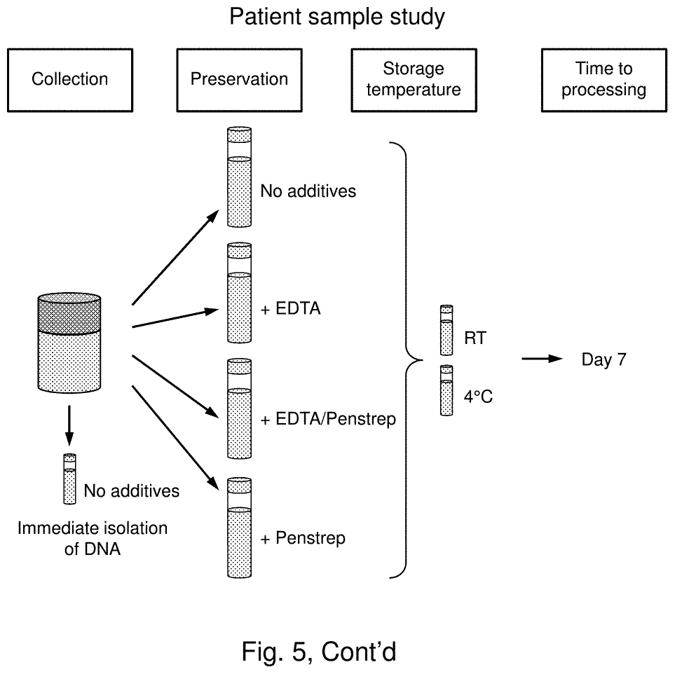

To preselect storage conditions for further testing on patient materials, the DNA yield for methylation analysis was determined in urine collected from three healthy volunteers stored under various conditions. All three volunteers provided written, informed consent to study participation. Each urine sample was divided into four equal volume aliquots of which one aliquot was used for immediate DNA isolation. DNA was extracted from native urine. Preserving agents, EDTA (final concentration of 40 mM) and 70 μl/ml Urine Conditioning Buffer™ (Zymo Research, Orange, Calif., U.S.A.), were added to aliquot two and three, respectively. No preserving agent was added to aliquot four. Thereafter samples were stored at different temperatures (room temperature (RT), 4° C., −20° C. and −80° C.) and processed on days 1, 2, 7 and 28 (FIG. 5).

Patient Sample Study

Urine samples were provided by bladder cancer and non-small cell lung cancer

(NSCLC) patients at the VU University Medical Center Amsterdam and the Amstelland Hospital Amstelveen between November 2016 and May 2017. Samples of bladder cancer patients (n=10) were collected prior to transurethral resection and samples from NSCLC patients prior to lobectomy (n=10). All specimens were divided into 9 equal volume aliquots. Preserving agents included EDTA in a final concentration of 40 mM and/or 20 ul/ml penicillin-streptomycin (PenStrep). Storage temperatures were RT or 4° C. Processing was done immediately after collection (day 0) and at day 7 (FIG. 5).

DNA Isolation

DNA was isolated using the Quick-DNA™ Urine Kit (Zymo Research, Orange, Calif., U.S.A.) according to the manufacturer's protocol. In the pilot study aliquots of 10 ml were used, whereas patient's samples contained equal aliquots of 4-10 ml, depending on the original volume. DNA was eluted in 50 μl of elution buffer and stored at −20° C.

Methylation Specific PCR

To allow for Quantitative Methylation Specific PCR (qMSP) analysis, 40 μl of isolated DNA was treated with bisulfite using the EZ DNA Methylation™ kit (Zymo Research, Orange, Calif., U.S.A.). DNA isolated from the bladder cancer cell line RT-112, kindly provided by prof. G. J. Peters (VU University Medical Center, Amsterdam, the Netherlands), was used for the generation of a standard curve. Quantitative MSPs of the housekeeping gene 6-actin (ACTB) and RASSF1A were performed as described previously [11, 12].

In short, amplification reactions contained a total volume of 12 μl including EpiTect MethyLight Master Mix (Qiagen), 200 nM of each primer and fluorescent dye-labeled probe, and 2.5 μl bisulfite treated DNA. The amplification reactions were carried out at 95° C. for 5 minutes, followed by 45 cycles at 95° C. for 15 seconds and 60° C. for 1 minute in 96-well plates in an ABI 7500 Fast Real-Time PCR System (Life Technologies, Thermofisher Scientific). Samples with ACTB cycle threshold (CT) of >32 (a commonly used threshold for defining unreliable hmDNA analysis) [13] at day 0 were excluded.

Data Analysis

For the pilot study a limited sample size (n=3) was used and no statistical comparisons were planned.

In the patients sample study, decay in DNA yield for hmDNA analysis was calculated by analyzing the log fold change of the ACTB (log 2FC_{ACTB}). The following formula was used: log 2FC_{ACTB}=log 2(2^{[CT}^{_{ACTB}}^{(t)-CT}^{_{ACTB}}^{(0)]}) (eq. 1) With CT_{ACTB}(t) and CT_{ACTB}(0) the CT values of ACTB at time t=7 days and day 0 respectively.

To define the correlation between ACTB-CT and RASSF1A-CT at various conditions the following formula was used:

ΔCT_{gene,condition X}=CT_{gene,condition X}(t)−CT_{gene,condition X}(0) (eq. 2)

With CT_{gene,condition X} (t) the CT of the gene of interest at time t with condition X. Prior to combining the samples of NSCLC and bladder cancer patients, differences in the ΔCT_{ACTB} and ΔCT_{RASSF1A} between both groups were analyzed using the Mann Whitney U test. When the differences were not statistically significant, samples of both groups were combined to increase statistical power. The difference between ΔCT_{ACTB} and ΔCT_{RASSF1A} at various conditions was assessed using Wilcoxon signed-rank tests. Differences between storage conditions were examined using a Friedman test for multiple extractions. When overall significance was observed, post-hoc Bonferroni-corrected analysis was performed using the related-samples Wilcoxon signed-rank test for two-by-two comparisons. The omnibus test for the multiple storage conditions was performed with R statistical software (version 3.3.1) using FSA package [14]. Remaining analyses were performed with SPSS software (SPSS 22.0, IBM, Armonk, N.Y., USA). All tests were two-sided and a significance level of 0.05 was applied.

Results

Pilot Study

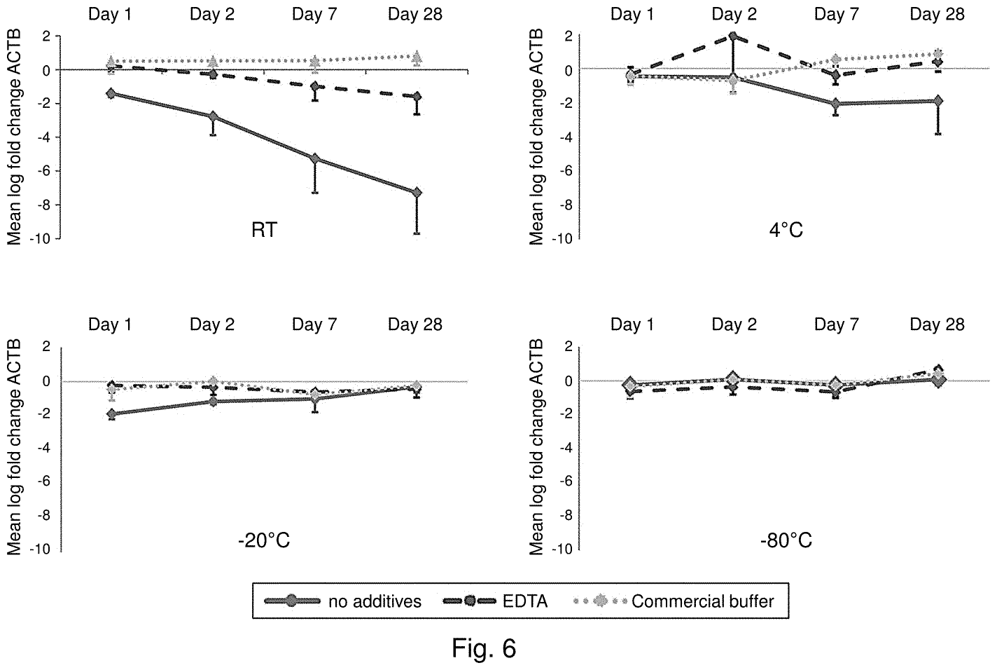

Analysis of three aliquoted urine samples stored at RT showed that the addition of both Urine Conditioning Buffer™ (Zymo Research, Orange, Calif., U.S.A.) and EDTA resulted in a better retained DNA yield as compared to no preserving agents (FIG. 6). None of the samples to which EDTA or Urine Conditioning Buffer™ was added had CT_{ACTB}>32. Without preserving agents, one out of three samples exceeded this value at day 2 and another one at day 7. At day 28 all three untreated samples had a CT_{ACTB}>32.

Upon storage at 4° C., DNA yield was maintained for the first two days, irrespective of the use of preserving agents (FIG. 6). After 7 days DNA yield was better retained if EDTA or the commercial buffer were added, as compared to no preserving agents (FIG. 6). At −20° C. and at −80° C. DNA yield was stable, regardless of the use of preserving agents (FIG. 6).

These results suggest that the addition of EDTA is similarly effective to the commercial buffer in terms of reducing DNA degradation when samples are stored at 4° C. At RT the effect of the commercial buffer and EDTA were comparable for a time period of 7 days. However, after 28 days the commercial buffer seemed superior to EDTA. Nevertheless, we chose to use EDTA for further testing for economic reasons. Furthermore, there was still a comparable efficacy at day 7, which is sufficient to allow for further processing. Storage at room temperature and 4° C. are the most practical in routine settings. For these reasons we chose to determine the efficacy of EDTA at RT and 4° C. in our further studies in which we used patient samples. In addition, with the possibility of bacterial contamination in mind, the addition of antibiotics (penicillin/streptomycin; PenStrep) was also tested.

Patient Sample Study

DNA Yield

One patient was excluded from analysis, due to a high CT value (>32) for ACTB at day 0. The differences in ΔCTACTB and ΔCTRASSF1A between NSCLC samples and bladder cancer samples did not significantly differ at any of the conditions (fable 11). As of such, samples of both patients groups were combined in further analyses to increase statistical power.

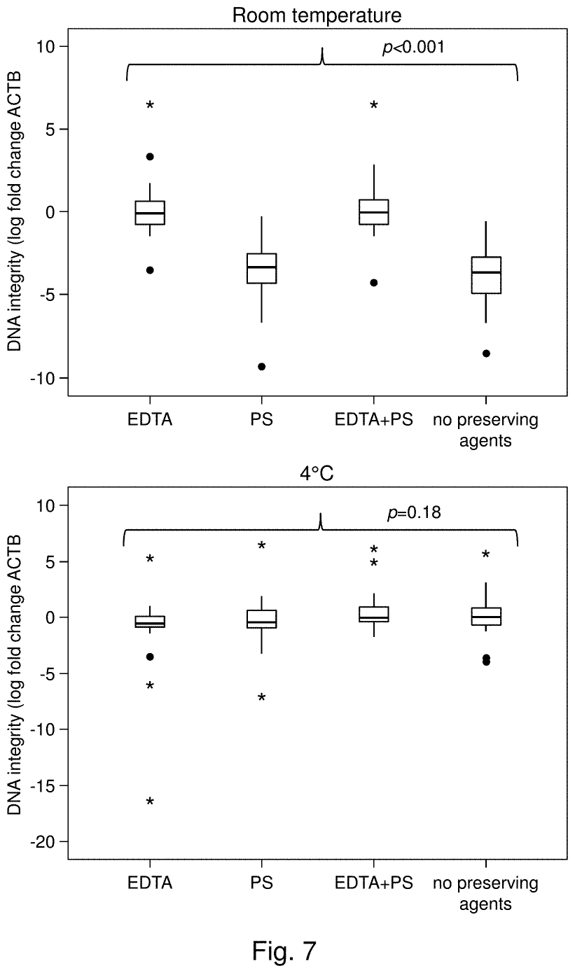

The calculated mean log fold change in ACTB levels (n=19) at various conditions are presented in FIG. 7. A statistically significant difference was present in the mean log fold change of ACTB level between the various conditions when urine samples were stored at RT (FIG. 7, p<0.001), but not when stored at 4° C. (FIG. 7, p=0.18).

A post-hoc analysis on differences in DNA yield for urine stored at RT demonstrated that DNA yield was significantly better when EDTA was added (FIG. 7). Addition of PenStrep did not improve DNA yield (p>0.99).

A total of seven samples had an CT_{ACTB}>32. Conditions of these samples included: PenStrep at RT (n=3), no preserving agents at RT (n=3) and PenStrep at 4° C. (n=1). None of the samples to which EDTA was added were associated with a CT_{ACTB}>32.

hmDNA Yield

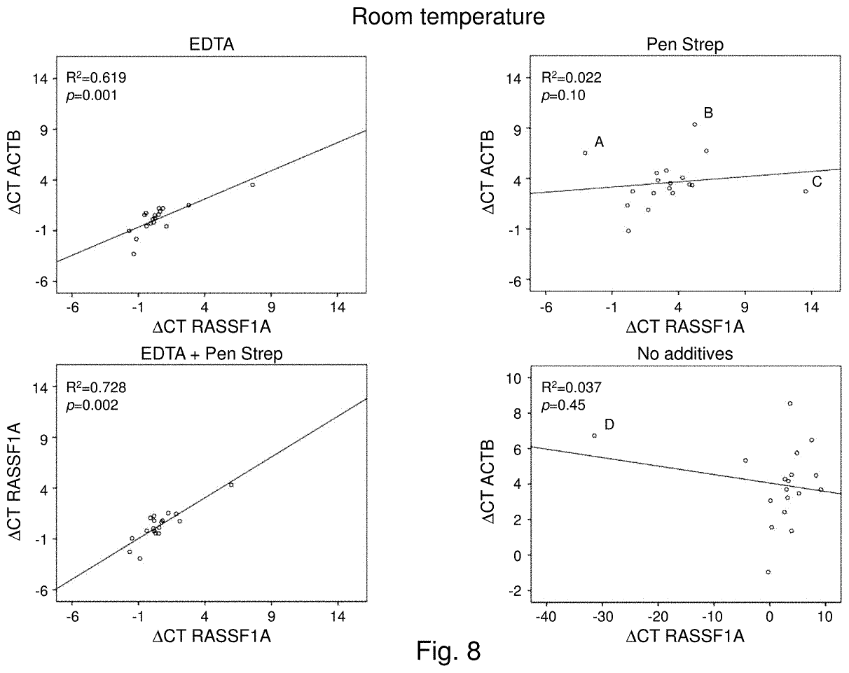

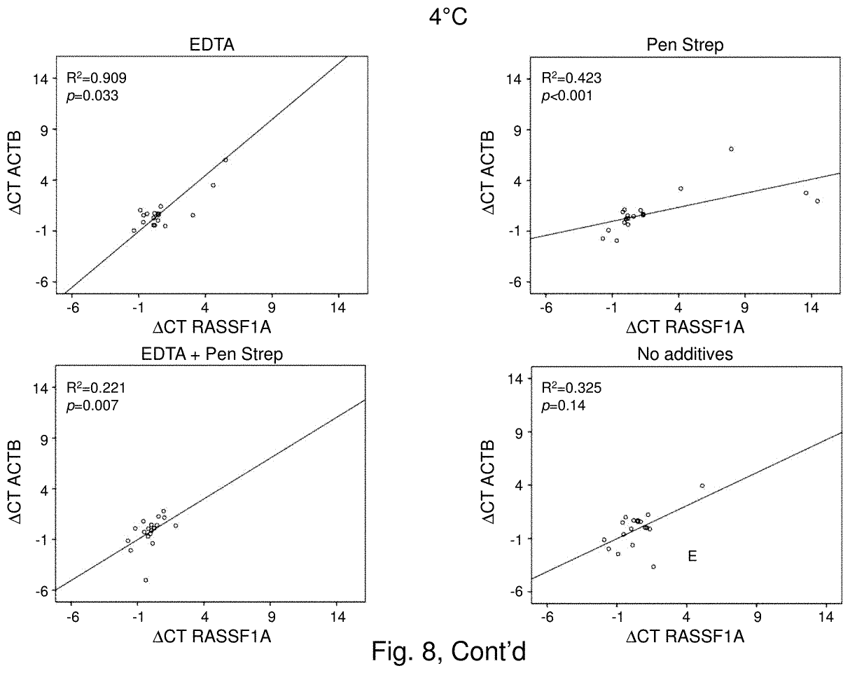

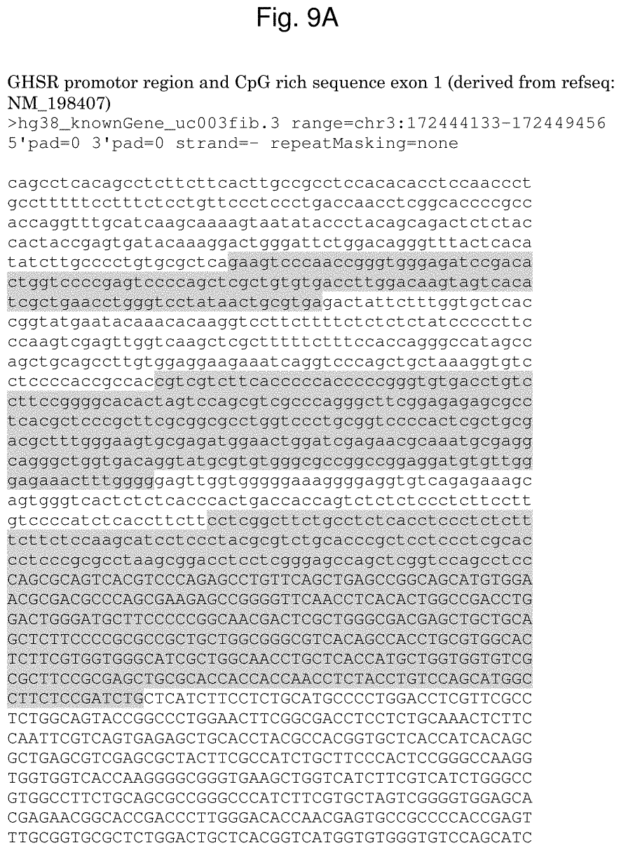

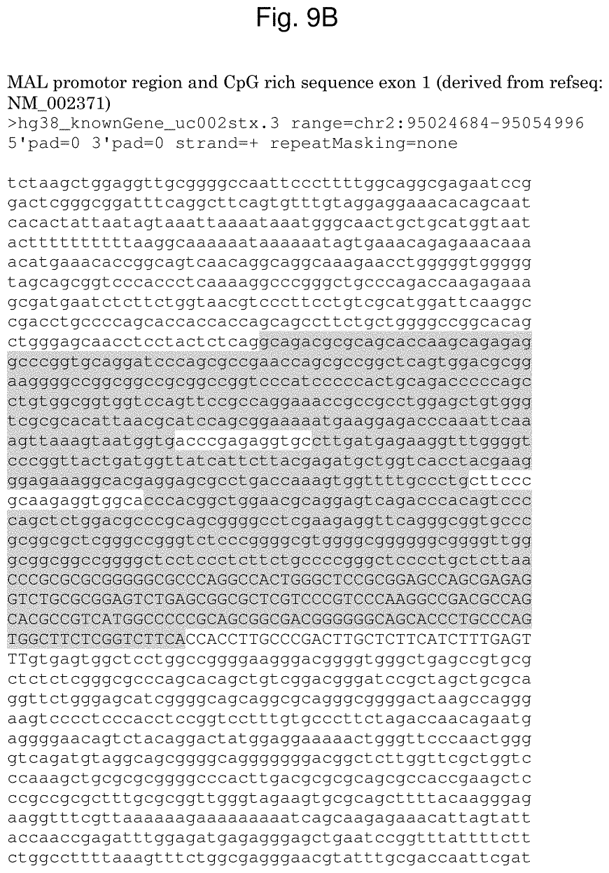

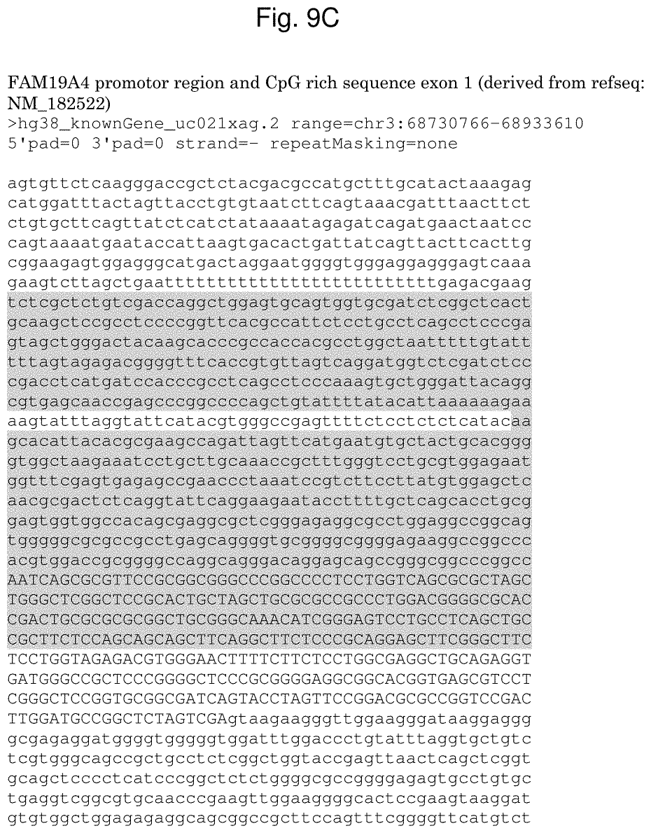

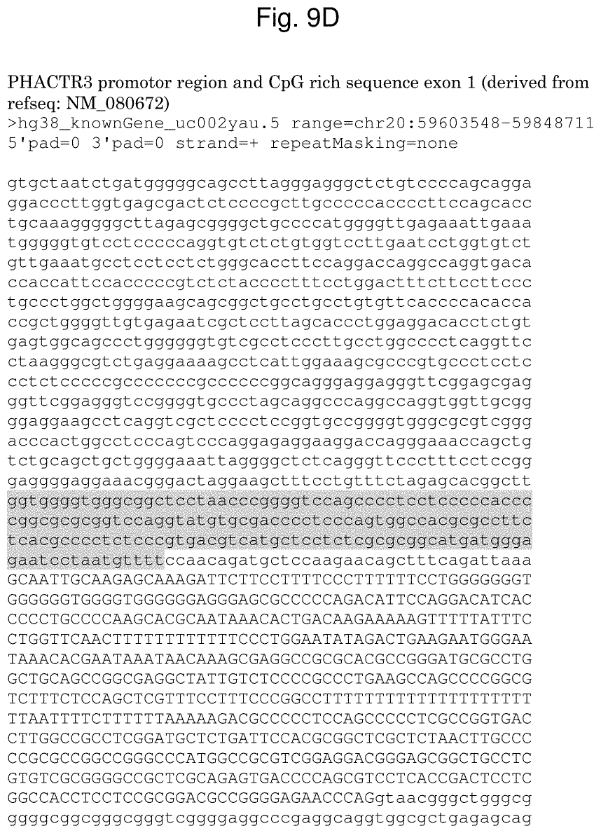

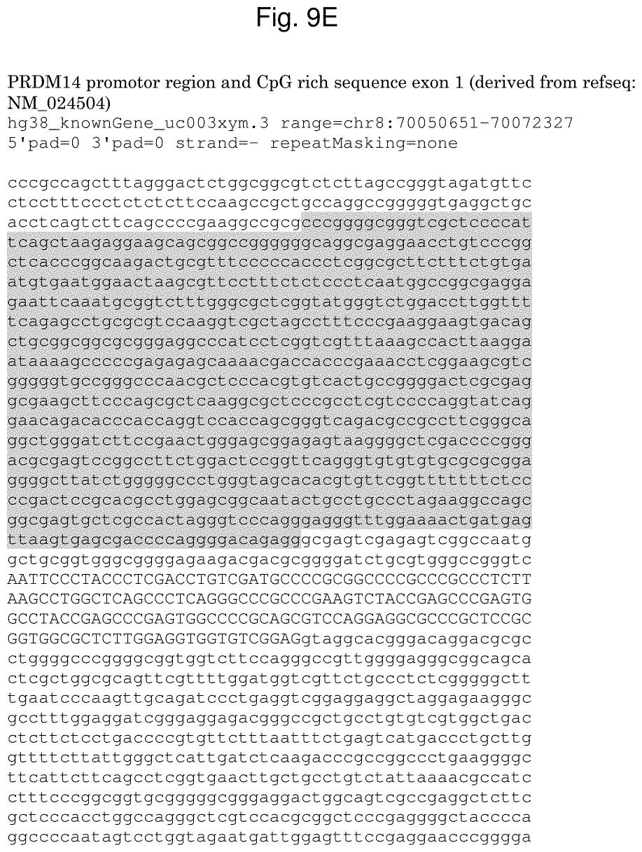

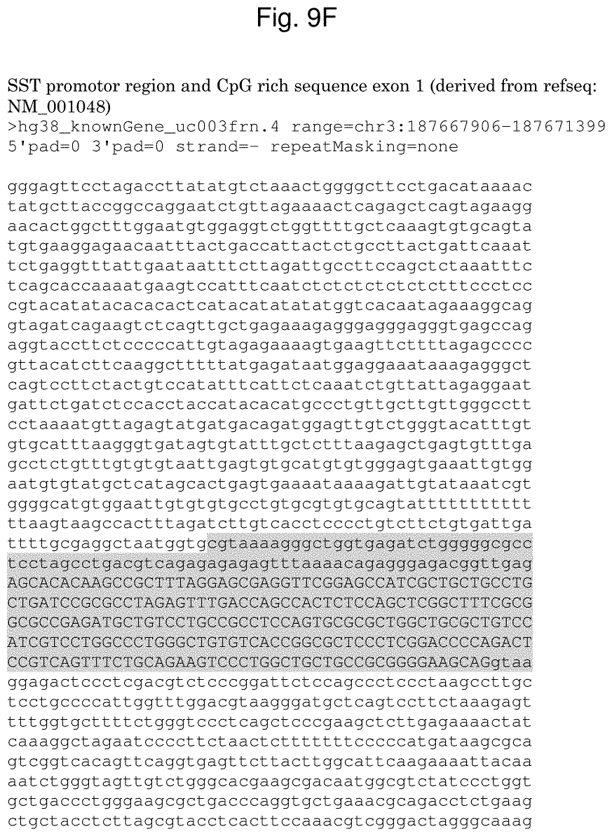

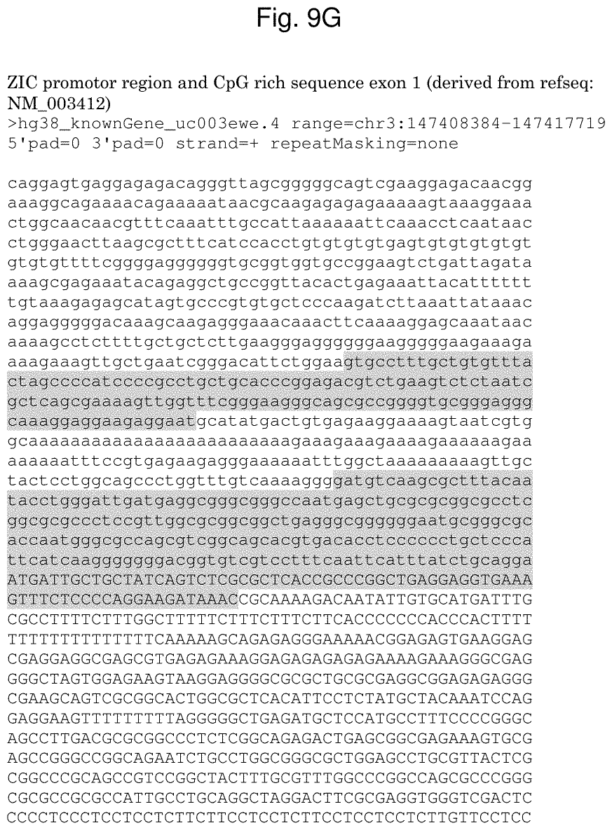

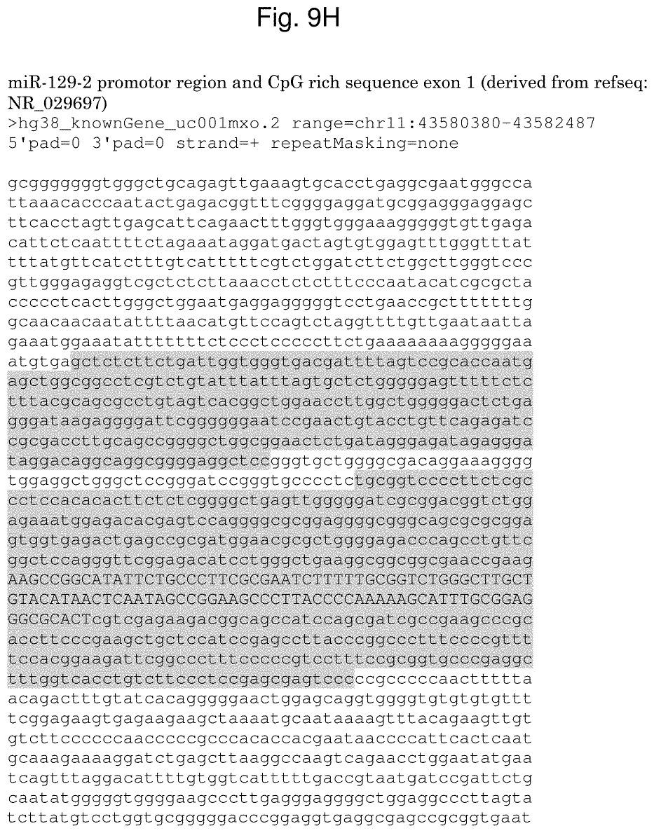

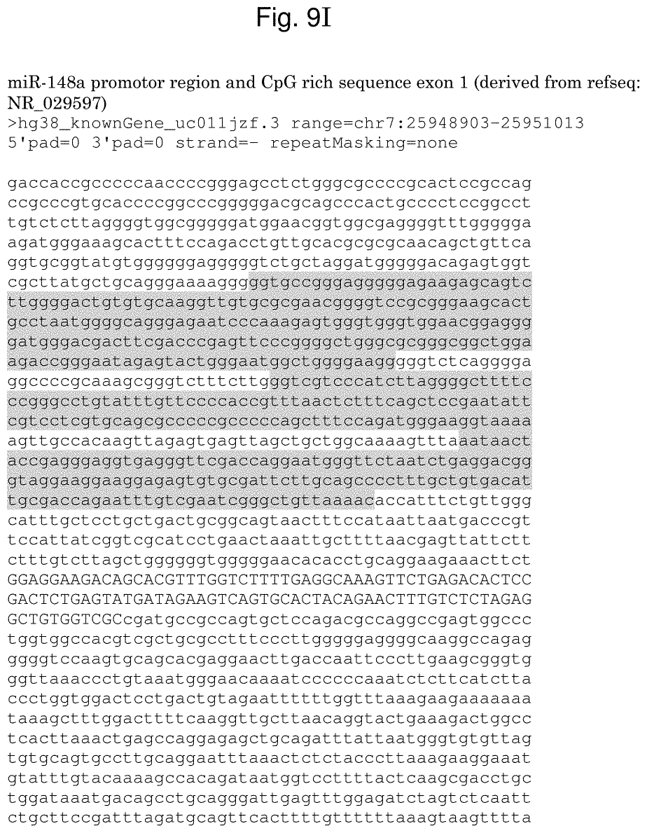

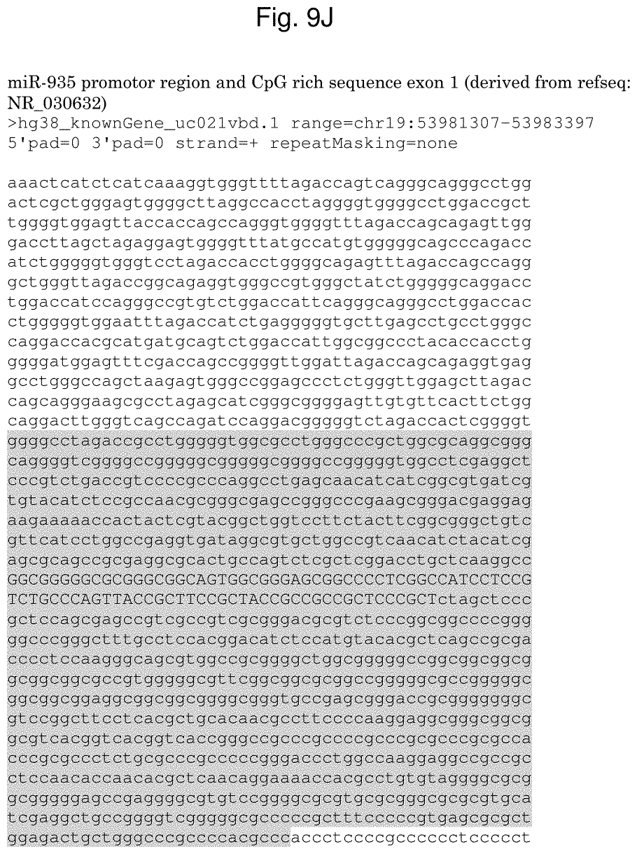

The difference between ΔCT_{ACTB} and ΔCT_{RASSF1A} at various conditions is Graphically presented in scatter plots (FIG. 8). There was no statistically significant difference in the ΔCT_{ACTB} and ΔCT_{RASSF1A} in any of the conditions (Table 12).

Five outliers, labeled A to E in FIG. 8, are the results of high CT values at which PCR quantification becomes inaccurate.

TABLE 11
CT difference of RASSF1A and ACTB at day 7 compared to day 0, in
samples of NSCLC patients versus bladder cancer patients at various conditions.

| | ΔCT_{ACTB} | | p-value | ΔCT_{RASSF1A} | | p-value |
|---|---|---|---|---|---|---|
| | median | | (Mann | median | | (Mann |
| | (IQR) | | Whitney | (IQR) | | Whitney |
| | BC | NSCLC | U test) | BC | NSCLC | U test) |
| Condition | | | | | | |
| at RT | | | | | | |
| EDTA | 0.57 | −0.24 | 0.2 | 0.16 | 0.16 | >0.9 |
| | (−0.22-1.05) | (−1.22-0.71) | | (−0.66-0.69) | (−0.85-0.83) | |
| PenStrep | 3.33 | 3.27 | 0.9 | 2.71 | 3.31 | 0.8 |
| | (1.73-4.66) | (2.25-4.69) | | (1.34-5.07) | (0.35-3.93) | |
| EDTA + | 0.64 | −0.10 | 0.6 | 0.23 | 0.19 | 0.8 |
| PenStrep | (−0.32-1.03) | (−1.28-1.19) | | (−0.52-0.75) | (−0.80-1.67) | |
| No | 4.28 | 3.46 | 0.4 | 3.33 | 3.19 | 0.7 |
| preserving | (2.42-6.24) | (2.21-4.70) | | (−0.22-4.16) | (0.23-7.91) | |
| agents | | | | | | |
| Condition | | | | | | |
| at 4° C. | | | | | | |
| EDTA | 0.55 | 0.44 | >0.9 | 0.15 | 0.48 | 0.2 |
| | (−0.28-0.73) | (−0.63-4.11) | | (−0.64-0.52) | (−0.38-5.06) | |
| PenStrep | 0.26 | 0.71 | 0.4 | 0.12 | 0.61 | 0.4 |
| | (−0.25-0.87) | (−1.11-2.87) | | (−0.23-1.19) | (−0.74-10.8) | |
| EDTA + | 0.08 | −0.03 | 0.7 | 0.01 | −0.19 | >0.9 |
| PenStrep | (−0.92-0.85) | (−1.37-0.49) | | (−0.67-0.33) | (−1.04-0.74) | |
| No | −0.10 | 0.28 | 0.6 | 0.07 | 0.50 | 0.5 |
| preserving | (−1.34-0.76) | (−1.34-0.76) | | (−0.67-1.08) | (−0.99-1.25) | |
| agents | | | | | | |

TABLE 12
The difference between the ACTACTB and ACTRASSF1A (day 7) for
various conditions.

| | ΔCT_{RASSF1A} | ΔCT_{ACTB} | p-value |
|---|---|---|---|
| | median | median | (Wilcoxon signed- |
| Condition at RT | (IQR) | (IQR) | rank test) |
| EDTA | 0.16 | 0.22 | 0.7 |
| | (−0.53-0.64) | (−0.58-0.90) | |
| PenStrep | 3.07 | 3.33 | 0.5 |
| | (0.55-4.81) | (2.55-4.54) | |
| EDTA + PenStrep | 0.19 | 0.11 | 0.2 |
| | (−0.39-0.80) | (−0.46-1.07) | |
| No preserving | 3.19 | 3.71 | 0.4 |
| agents | (0.13-4.88) | (2.42-5.33) | |
| | ΔCT_{RASSF1A} | ΔCT_{ACTB} | p-value |
| | median | median | (Wilcoxon signed- |
| Condition at 4° C. | (IQR) | (IQR) | rank test) |
| EDTA | 0.21 | 0.55 | 0.4 |
| | (−0.63-0.99) | (−0.42-1.05) | |
| PenStrep | 0.19 | 0.53 | 0.16 |
| | (−0.21-1.33) | (−0.35-1.12) | |
| EDTAP + PenStrep | −0.03 | 0.05 | >0.9 |
| | (−0.56-0.46) | (−1.13-0.44) | |
| No preserving | 0.20 | −0.01 | 0.12 |
| agents | (−0.59-1.15) | (−1.62-0.69) | |

### Example 3 Different Urine Samples

The data was based on the analysis of DNA methylation in native (full) urine samples of patients with bladders cancer and age matched healthy controls. The methylation markers for bladder cancer detection have now been compared on different urine fractions. We compared urine sediment, to urine supernatant and full (native) urine using urine of 14 bladder cancer patients and 12 matched controls with benign hematuria. The latter population represents the population frequently seen at a urology clinic, that is in highest need to be distinguished from bladder cancer patients.

We found that the use of urine sediment significantly (p<0.05) increased the sensitivity of many of the markers of table 4 both individually as well as in combination. The use of urine sediment significantly (p<0.05) increased the sensitivity of the markers GHSR and MAL and the combination GHSR/MAL for bladder cancer detection compared to the use of full urine. The relative sensitivity was 1.60 (GHSR), 1.50 (MAL) and 1.43 (GHSR/MAL) respectively. This finding was particularly surprising given the fact that no increase in sensitivity was found for some of the other markers such as FAM19A4, SST and ZIC1 when comparing their diagnostic performance in urine sediment to full urine. The sensitivity of the markers GHSR and MAL and the combination GHSR/MAL for bladder cancer increased most when compared to the other markers and combinations of two thereof in table 4.

We next determined the correlation between the methylation levels detected in urine sediment to the methylation levels detected in the tumor tissues of the same patients. This analysis revealed that GHSR and MAL showed the highest correlation between urine sediment and tissue specimens (Spearman's correlation 0.768 (GHSR) and 0.744 (MAL). In comparison FAM19A4 showed a correlation of 0.567, SST of 0.675 and ZIC1 of 0.691.

These findings demonstrate that the diagnostic accuracy of GHSR, MAL and the panel GHSR/MAL increases when urine sediment is tested. Above all, these markers appeared to most closely reflect the methylation status in the tumor itself. These findings demonstrate the surprisingly superior performance of GHSR, MAL and GHSR/MAL above other methylation markers.

### REFERENCES

Chan M W, Chan L W, Tang N L et al. Hypermethylation of multiple genes in tumor tissues and voided urine in urinary bladder cancer patients. Clin Cancer Res 8(2), 464-470 (2002).
Chou R, Gore J L, Buckley D et al. Urinary Biomarkers for Diagnosis of Bladder Cancer: A Systematic Review and Meta-analysis. Ann Intern Med 163(12), 922-931 (2015).
De Strooper L M, Van Zummeren M, Steenbergen R D et al. CADM1, MAL and miR124-2 methylation analysis in cervical scrapes to detect cervical and endometrial cancer. J Clin Pathol 67(12), 1067-1071 (2014).
Hubers A J, Heideman D A, Burgers S A et al. DNA hypermethylation analysis in sputum for the diagnosis of lung cancer: training validation set approach. Br J Cancer 112(6), 1105-1113 (2015).
Marshall R. The predictive value of simple rules for combining two diagnostic tests. Biometrics 1213-1222 (1989).
Schmittgen T D, Livak K J. Analyzing real-time PCR data by the comparative C(T) method. Nat Protoc 3(6), 1101-1108 (2008).
Schisterman E F, Perkins N J, Liu A, Bondell H. Optimal cut-point and its corresponding Youden Index to discriminate individuals using pooled blood samples. Epidemiology 16(1), 73-81 (2005).
Steenbergen R D, Ongenaert M, Snellenberg S et al. Methylation-specific digital karyotyping of HPV16E6E7-expressing human keratinocytes identifies novel methylation events in cervical carcinogenesis. J Pathol 231(1), 53-62 (2013).
Verlaat W, Snijders PJF, Novianti P W et al. Genome-wide DNA Methylation Profiling Reveals Methylation Markers Associated with 3q Gain for Detection of Cervical Precancer and Cancer. Clin Cancer Res 23(14), 3813-3822 (2017).
Wilting S M, Miok V, Jaspers A et al. Aberrant methylation-mediated silencing of microRNAs contributes to HPV-induced anchorage independence. Oncotarget 7(28), 43805-43819 (2016).
Youden W J. Index for rating diagnostic tests. Cancer 3(1), 32-35 (1950).

1. A method for classifying an individual as having bladder cancer or recurrent bladder cancer or being at risk of developing bladder cancer, the method comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935 and classifying said individual based on said DNA methylation.

2. Method according to claim 1 comprising comparing said DNA methylation with at least a first and a second reference value and classifying said individual based on said comparison.

3. Method according to claim 1, wherein said urine sample comprises a full urine sample or comprises cells or DNA isolated from urine.

4. Method according to claim 1, wherein DNA methylation of promoter regions of at least GHSR and MAL is determined.

5. Method according to claim 1, wherein DNA methylation of promoter regions is determined of:
at least GHSR and FAM19A4,
at least GHSR and PHACTR3,
at least GHSR and PRDM14,
at least GHSR and SST,
at least GHSR and miR-129,
at least GHSR and miR-148,
at least GHSR and miR-935,
at least SST and MAL, or
at least SST and PRDM14.

6. Method according to claim 1, comprising:
isolating DNA;
treating isolated DNA with bisulphite;
performing quantitative methylation-specific PCR (qMSP).

7. Method according to claim 1, wherein the bladder cancer is non-muscle-invasive bladder cancer (NMIBC) or muscle-invasive bladder cancer (MIBC).

8. Method according to claim 1, wherein said urine sample has been treated with a preservative, preferably ethylenediaminetetraacetic acid (EDTA), and/or an antibiotic.

9. A method for typing a urine sample from an individual, the method comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935 and typing said urine sample on the basis of DNA methylation.

10. A method for determining a treatment strategy for an individual, comprising determining DNA methylation of at least a first gene or a promoter region thereof and a second gene or a promoter region thereof in a urine sample from said individual, wherein said genes are selected from the group consisting of GHSR, MAL, FAM19A4, PHACTR3, PRDM14, SST, ZIC1, miR-129, miR-148 and miR-935, and determining a treatment strategy for said individual if determined DNA methylation indicates that said individual is having bladder cancer or recurrent bladder cancer or at risk of having bladder cancer.

11. A kit of parts comprising means for the detection of DNA methylation in at least a first gene or a promoter region thereof and a second gene or a promoter region thereof, wherein said genes are selected from the group consisting of GHSR, MAL, FAM19A4, PHACTR3, PRDM14, SST, ZIC1, miR-129, miR-148 and miR-935.

12. Use of kit of parts according to claim 11 for classifying an individual as having bladder cancer or recurrent bladder cancer or being at risk of developing bladder cancer.

13. Use of a urine sample comprising a preservative, preferably ethylenediaminetetraacetic acid (EDTA), and optionally an antibiotic for analysis of DNA methylation of one or more genes or a promoter region thereof.

14. Use according to claim 13 whereby said urine sample is stored for at least one week prior to said analysis.
